SEQUENCE LISTING (29 sequences)

SEQ ID NO: 1 - DNA, 27 bp, Artificial Sequence
  Synthetic i-motif
ttttttcccttacccttacccttaccc

SEQ ID NO: 2 - DNA, 35 bp, Artificial Sequence
  Synthetic i-motif
ttttttcccccttacccccttacccccttaccccc

SEQ ID NO: 3 - DNA, 43 bp, Artificial Sequence
  Synthetic i-motif
ttttttcccccccttacccccccttacccccccttaccccccc

SEQ ID NO: 4 - DNA, 51 bp, Artificial Sequence
  Synthetic i-motif
ttttttcccccccccttacccccccccttacccccccccttaccccccccc

SEQ ID NO: 5 - DNA, 35 bp, Artificial Sequence
  Synthetic i-motif
caccactcctcactcctctcctctctcacaccttc

SEQ ID NO: 6 - DNA, 21 bp, Artificial Sequence
  Synthetic primer
cgagcagagatcttcaggaac

SEQ ID NO: 7 - DNA, 20 bp, Artificial Sequence
  Synthetic primer
tcactaccagtcccagagtc

SEQ ID NO: 8 - DNA, 22 bp, Artificial Sequence
  Synthetic primer
ctttgtcaagctcatttcctgg

SEQ ID NO: 9 - DNA, 19 bp, Artificial Sequence
  Synthetic primer
tcttgctcagtgtccttgc

SEQ ID NO: 10 - DNA, 22 bp, Artificial Sequence
  Synthetic primer
ggtgcctatgtctcagcctctt

SEQ ID NO: 11 - DNA, 23 bp, Artificial Sequence
  Synthetic primer
gccatagaactgatgagagggag

SEQ ID NO: 12 - DNA, 22 bp, Artificial Sequence
  Synthetic primer
atcatccctgcgagcctatcct

SEQ ID NO: 13 - DNA, 24 bp, Artificial Sequence
  Synthetic primer
gaccttttttggctaaacgctttc

SEQ ID NO: 14 - DNA, 24 bp, Artificial Sequence
  Synthetic primer
cctagtgataaggaatgcacgatg

SEQ ID NO: 15 - DNA, 23 bp, Artificial Sequence
  Synthetic primer
ctaggcaggtttgatctccgttc

SEQ ID NO: 16 - DNA, 21 bp, Artificial Sequence
  Synthetic primer
tcccagtgaatactgatgaga

SEQ ID NO: 17 - DNA, 20 bp, Artificial Sequence
  Synthetic primer
ccactctggatctcccaaga

SEQ ID NO: 18 - DNA, 20 bp, Artificial Sequence
  Synthetic primer
gggcatacctttatcctgag

SEQ ID NO: 19 - DNA, 20 bp, Artificial Sequence
  Synthetic primer
ccactgaagtcatccatgtc

SEQ ID NO: 20 - DNA, 20 bp, Artificial Sequence
  Synthetic primer
cagaagaatggaagagtcag

SEQ ID NO: 21 - DNA, 20 bp, Artificial Sequence
  Synthetic primer
cagatatgcagggagtcacc

SEQ ID NO: 22 - DNA, 24 bp, Artificial Sequence
  Synthetic primer
tgcatggaccagtataaggcaagc

SEQ ID NO: 23 - DNA, 24 bp, Artificial Sequence
  Synthetic primer
gcttctggtcgatgtcatgagcaa

SEQ ID NO: 24 - DNA, 23 bp, Artificial Sequence
  Synthetic primer
gttcacctggagtgatggttctc

SEQ ID NO: 25 - DNA, 22 bp, Artificial Sequence
  Synthetic primer
aggacatgccagggtcaccttt

SEQ ID NO: 26 - DNA, 23 bp, Artificial Sequence
  Synthetic primer
gaggataccactcccaacagacc

SEQ ID NO: 27 - DNA, 24 bp, Artificial Sequence
  Synthetic primer
aagtgcatcatcgttgttcataca

SEQ ID NO: 28 - DNA, 25 bp, Artificial Sequence
  Synthetic primer
cagaagctaaccatctcctggtttg

SEQ ID NO: 29 - DNA, 25 bp, Artificial Sequence
  Synthetic primer
tccggagtaatttggtgcttcacac

(19) United States
(12) Patent Application Publication
(10) Pub. No.: US 2024/0148900 A1
(43) Pub. Date: May 9, 2024

(54) IMMUNOSTIMULATORY CYCLIC DI-NUCLEOTIDE DELIVERY SYSTEM COMPOSITIONS AND METHOD OF USE THEREOF

(71) Applicant: VIRGINIA COMMONWEALTH UNIVERSITY, Richmon, VA (US)

(72) Inventors: Guizhi ZHU, Glen Allen, VA (US); Yu ZHANG, Richmond, VA (US)

(21) Appl. No.: 18/281,108

(22) PCT Filed: Apr. 14, 2022
(86) PCT No.: PCT/US2022/024823

§ 371 Date: Sep. 8, 2023

Related U.S. Application Data
(60) Provisional application No. 63/175,083, filed on Apr. 15, 2021.

(51) Int. Cl. A61K 47/69; A61K 47/54; A61P 35/00

(52) U.S. Cl. CPC A61K 47/6937; A61K 47/549; A61P 35/00

(57) ABSTRACT

Provided herein are pH-responsive nanovaccines (NVs) that stimulate the immune system. The NVs comprise nano- or micro-particles to which CDN-modified i-motif DNA is attached. When endocytosed within a subject to whom they are delivered, the change in pH to an acidic environment cause the CDNs to be released from the NVs. The CDNs are STING (stimulator of interferon genes) agonists and after their release, they bind to and activate STING, a critical step in stimulating the immune system. The NVs are used e.g. for cancer immunotherapy, to treat viral infections and/or as vaccine adjuvants.

### CROSS-REFERENCE TO RELATED APPLICATIONS

This application claims the benefit of U.S. patent application 63/175,083, filed Apr. 15, 2021.

### STATEMENT OF FEDERALLY SPONSORED RESEARCH AND DEVELOPMENT

This invention was made with government support under grant R21 NS114455 awarded by the National Institutes of Health/the National Institute of Neurological Disorders and Stroke. The United States government has certain rights in the invention.

### SEQUENCE LISTING

This application includes as the Sequence Listing the complete contents of the accompanying text file “Sequence.txt”, created Apr. 9, 2022, containing 8192 bytes, hereby incorporated by reference.

### BACKGROUND OF THE INVENTION

### Technical Field

The invention generally relates to pH-responsive nanovaccines (NVs) that stimulate the immune system. In particular, the invention provides NVs comprising nano- or micro-particles to which CDN-modified i-motif DNA is attached, and methods of using the NVs to deliver CDNs to cells in order to stimulate the immune system by activating the STING (stimulator of interferon genes) protein.

### Description of Related Art

The past decade has witnessed the historical breakthrough of cancer immunotherapy.^{[1]} However, current immunotherapy approaches, especially immune checkpoint blockade, have only benefited a small subset of cancer patients.⁴ Immunostimulants hold great potential to elicit or augment antitumor immune responses, ameliorate immunosuppression systemically and locally in the tumor microenvironment (TME), and synergize or complement current immunotherapy approaches.^{[2]} STING (stimulator of interferon genes) agonists are a novel class of immunostimulants.^{[3]} STING, also known as transmembrane protein 173 (TMEM173), is a cytosolic DNA sensor and an adaptor protein that primarily resides in the endoplasmic reticulum of a wide variety of immune cells, and STING activation has been involved in microbial infection, autoimmune diseases, and cell senescence.^{[3a]} Natural STING agonists that are directly or indirectly derived from viruses, bacteria, protozoa, and dying self-cells can activate the STING signaling pathway and hence upregulate the expression of a variety of pro-inflammatory cytokines and chemokines, including type I interferons (IFNs), in a wide range of cells such as macrophages, dendritic cells (DCs), monocytes, myeloid derived suppressor cells (MDSCs), as well as cancer cells.^{[4a, 4b]} STING agonists, such as CDNs (cyclic dinucleotides), have been developed and tested for cancer, coronavirus and influenza immunotherapy.^{[5]} Naturally derived CDNs are particularly attractive as candidates for active innate immunity, such as cyclic dimeric guanosine monophosphate (CDG), cyclic dimeric adenosine monophosphate (CDA), and cyclic GMP-AMP (cGAMP).^{[6]} In addition, it has been reported that coordinated antigen presentation and STING agonist activation can mediate adaptive immunity development.^{[5d, 5e, 5f]} Although these CDNs, typically administered intralesionally, have been investigated for cancer immunotherapy, the development of CDN immunotherapeutics has been hampered by the poor pharmacokinetics given their hydrophilicity, small molecular sizes, negative charges, and susceptibility to enzymatic degradation.^{[3c, 4c]} Current approaches to address these complications include chemical modifications that enhance the biostability of CDNs^{[7]}, and drug delivery systems based on passive loading or electrostatic loading into liposome^{[5f, 8]}, polymer^{[5d]}, polymersome^{[5e, 9]}, inorganic nanoparticles (NPs)^{[10]}, and hydrogels^{[11]}. However, there is a need to further develop strategies to overcome the complications that hamper the development of CDN immunotherapeutics.

### SUMMARY OF THE INVENTION

Other features and advantages of the present invention will be set forth in the description of invention that follows, and in part will be apparent from the description or may be learned by practice of the invention. The invention will be realized and attained by the compositions and methods particularly pointed out in the written description and claims hereof.

Cyclic dinucleotides (CDNs), such as c-di-GMP (CDG), are agonists for stimulator of interferon genes (STING) and are promising for cancer immunotherapy. Yet, the therapeutic efficacy of CDNs has been limited by poor delivery and biostability. This disclosure describes STING-activating DNA nanovaccines (STING-NVs) that bio-stabilized, delivered, and conditionally released CDG in the endosome of immune cells for cancer immunotherapy. STING-NVs had nanoparticle scaffolds and cytosine (C)-rich i-motif DNA immobilized on the scaffold. I-motif DNA underwent a characteristic pH-responsive conformational switch, allowing efficient CDG loading via C:G base pairing at physiological pH and CDG release under acidic pH. STING-NVs protected CDG from enzymatic degradation. STING-NVs facilitated cell delivery and endosome escape of CDG, and potentiated antitumor immunity. STING-NVs repolarized immunosuppressive M2-like macrophages into antitumor M1-like macrophages in vitro and in vivo. Intralesional STING-NVs outperformed liposomal CDG and fluoride-CDG for melanoma immunotherapy. pH-responsive STING-NVs are thus effective agents for cancer immunotherapy.

It is an object of this invention to provide a drug delivery system for delivering stimulator of interferon genes (STING) agonists, comprising nano- or microparticulate scaffolds; synthesized cytosine-rich i-motif DNA on the nano- or microparticulate scaffolds; and cyclic dinucleotides (CDNs) bound to the cytosine-rich i-motif DNA. In some aspects, the nano- or microparticulate scaffolds comprise poly(D,L-lactide)-block-poly(ethylene glycol). In further aspects, the cytosine-rich i-motif DNA comprises four cytosine-rich domains. In additional aspects, the cytosine-rich i-motif DNA comprises 3-9 consecutive cytosines in each cytosine-rich domain. In yet further aspects, the CDNs comprise one or more of cyclic dimeric guanosine monophosphate (CDG), cyclic dimeric adenosine monophosphate (CDA), and cyclic GMP-AMP (cGAMP). In additional aspects, a molar ratio of polymer in the nano- or microparticulate scaffolds to the cytosine-rich i-motif DNA is from 18:1 to 22:1. In other aspects, the nano- or microparticulate scaffolds have an average diameter of 70-100 nm. In further aspects, the cytosine-rich i-motif DNA is configured to release the CDNs at a pH of 6.8 or lower.

Also provided is a method for delivering STING agonists to a subject in need thereof, comprising administering to the subject a therapeutically effective amount of the drug delivery system disclosed herein, i.e. comprising nano- or microparticulate scaffolds; synthesized cytosine-rich i-motif DNA on the nano- or microparticulate scaffolds; and cyclic dinucleotides (CDNs) bound to the cytosine-rich i-motif DNA. In some aspects, the subject has cancer. In further aspects, the cancer is melanoma. In additional aspects, the subject has a viral infection.

### BRIEF DESCRIPTION OF THE DRAWINGS

FIG. 1A-H. Characterization of STING-NVs. (a) UV-vis absorbance spectra of PEG-b-PLA NPs, 5c-i-motif DNA, and 5c-NPs. (b) An agarose electrophoresis image that showed conjugation of i-motifs on NPs. Lane legends: 1: 25 bp DNA ladder; 2: 9C-i-motif; 3: 3C-NPs; 4: 5C-NPs; 5: 7C-NPs; 6: 9C-NPs. (c) DLS graphs showing the hydrodynamic diameters of 5C-NPs (79±34 nm, PDI 0.157) and STING-NVs (81±32 nm, PDI 0.131). (d) A TEM image of STING-NVs. (e) Zeta potential of blank NPs, CDG-loaded NPs, and Lipo-CDG. STING-NVs: CDG-loaded 5C-NPs. (f) Stability of free CDG and CDG loaded in STING-NVs over 7-day incubation in 10% FBS-supplemented cell culture medium (37° C.). A₂₅₄ₙₘ: 254 nm absorption from CDG; A₅₆₀ₙₘ: 560 nm absorption from medium as an internal reference. (g, h) MST traces (g) and fitting curve (h) of the kinetic interaction between 5C-NPs and CDG at pH 7.4. Data represent mean±standard deviation (SD) (n=3).

FIG. 2. pH-responsive cumulative CDG release from STING-NVs. Data represent mean±SD (n=3).

FIG. 3A-C. Efficient intracellular uptake and endosome escape of STING-NVs in macrophages. (a) Flow cytometry results of RAW264.7 macrophages treated with free CDG, Lipo-CDG, STING-NVs, respectively, for 0.5, 1 and 2 h. (b) CLSM images of RAW264.7 cells treated with the above formulations for 1 h. Blue: nuclei stained with Hoechst33342. Red: endolysosome stained with LysoTracker Red DND-99. Green: FluoCDG. Insets: close-up views of single cells. (c) The signal ratio of FluoCDG outside/inside (O/I) endolysosome, which was stained using Lysotracker Red in RAW264.7 cells after treatment for 1 h. FluoCDG: 0.5 μg/mL CDG equiv. Data represent mean±SD (n=10). ****p<0.0001, by Student's t-test.

FIG. 4A-E. STING-NVs promoted the conversion of M2-like macrophages into M1-like macrophages. (a) Murine RAW264.7 macrophages were treated with LPS and IFN-γ to induce an M1-like phenotype and were treated with IL-4 to induce an M2-like phenotype. UT Mφ, untreated macrophage. On day 2, M2 cells were treated with free CDG, F-CDG, 5C-NPs, STING-NVs, or Lipo-CDG, respectively (1.5 μg/mL CDG equiv.) (n=3). (b) ELISA analysis of IL-6, IL-12 and IFN-α levels in supernatants collected on day 3. (c) On day 3, treated cells were analyzed by flow cytometry for the levels of M1 markers (I-A/I-E and CD86) and M2 marker (CD206). (d) On day 3, treated cells were lysed for the RT-qPCR analysis of mRNA levels of M2 markers (Ym1, Fizz1, Arg1, and Mrc1) and (e) M1 markers (NOS2, Tnf, IL6, and IL12). N.D.: not determined. Data represent mean±SD. **p<0.01; ***p<0.001; ****p<0.0001, by one-way ANOVA with a Tukey's post hoc test (b-e; vs. STING-NVs).

FIG. 5. Potent immunostimulation by STING-NVs in human THP-1 monocytes. Shown are the dose-responsive IFN-α/β production after treatment with STING-NVs and controls for 24 h, as measured using HEK-Blue™ IFN-α/β reporter cells. Absorption: 630 nm.

FIG. 6A-G. STING-NVs remodeled the tumor immune microenvironment for potent immunotherapy. (a) Study scheme using B16F10 melanoma in syngeneic C57BL/6 mice. When tumor reached ˜60 mm³, mice were intratumorally administered with PBS, F-CDG, 5C-NPs, Lipo-CDG, or STING-NVs, respectively every three days for five times (dose: 3 μg CDG equivalents). (b-d) 3 days after the last treatment (day 22), RT-qPCR was used to analyze the transcript levels of (b) M2 gene markers (Ym1, Arg1, and Mrc1), (c) M1 gene markers (Tnf, Il6, and Nos2), and (d) antitumor markers Ifnb1, Cxcl9 and Cxcl10 in B16F10 tumors (n=5). (e-g) After the same treatment as above, tumor volumes (e), mouse survival (f), and mouse body weights (g) were monitored (n=5-10 biologically independent samples, respectively). Data represent mean±SD (b-d) or mean±s.e.m. (e, g). *p<0.05; **p<0.01; ***p<0.001, by one-way ANOVA with a Tukey's post hoc test (b-g; vs. STING-NVs).

FIG. 7A-C. Size characterization of NPs. (a) DLS graphs showing the hydrodynamic diameters of PEG-b-PLA NPs (NPs, 78±35 nm, PDI 0.195), and NPs modified with 3C-i-motif (3C-NPs, 82±28 nm, PDI 0.125), 5C-i-motif (5C-NPs, 79±34 nm, PDI 0.157), 7C-i-motif (7C-NPs, 83±38 nm, PDI 0.201), and 9C-i-motif (9C-NPs, 84±41 nm, PDI 0.212). (b) DLS graphs of CDG loaded 3C-NPs (CDG-3C-NPs), CDG loaded 5C-NPs (CDG-5C-NPs), CDG loaded 7C-NPs (CDG-7C-NPs), and CDG loaded 9C-NPs (CDG-9C-NPs). The hydrodynamic diameters of CDG-3C-NP, CDG-5C-NP, CDG-7C-NP, and CDG-9C-NP are 79±40 nm, 81±32 nm, 81±36 nm, 82±35 nm, and 88±45 nm, respectively. The PDI of CDG-3C-NPs, CDG-5C-NPs, CDG-7C-NPs, and CDG-9C-NPs are 0.215, 0.131, 0.147, 0.225, and 0.231, respectively. (c) DLS of CDG-loaded liposome (Lipo-CDG). The hydrodynamic diameters of Lipo-CDG are 131±18 nm. The PDI of Lipo-CDG is 0.057.

FIG. 8A-C. UV-vis absorption spectra of PEG-b-PLA NPs, DNA i-motif, and i-motif-NPs for A, 3C-i-motifs, B, 7C-i-motifs, and C, 9C-i-motifs, respectively.

FIG. 9. Zeta potential of NPs, 3C-NPs, 5C-NPs, 7C-NPs, and 9C-NPs. Data represent mean±standard deviation (SD) (n=3).

FIG. 10. HPLC diagrams of free CDG, 5C-i-motif, and a physical mixture of CDG and 5C-i-motif (molar ratio: 2:1) at pH 7.4.

FIG. 11A-D. Microscale thermophoresis (MST) analysis of the kinetic association and dissociation between CDG and scramble DNA-NPs at pH 7.4 (a-b), and between CDG and 5C-NPs at pH 5.0 (c-d). (a, c) MST traces, (b, d) MTS quantification.

FIGS. 12A and B. In vitro release profiles of CDG from (a) 3C-NPs, 5C-NPs, 7C-NPs, and 9C-NPs, as well as (b) Lipo-CDG at pH 5.0 and 7.4. Data represent mean±SD (n=3).

FIG. 13. RAW264.7 macrophages were cultured in LPS plus IFN-γ for 24 hours to induce an M1-like phenotype and IL-4 to induce an M2-like phenotype. UT M ϕ, untreated control. On day 2, M2 cells were treated with 1.5 μg/mL CDG delivered as free CDG, F-CDG, 5C-NPs, STING-NVs or Lipo-CDG. On day 3, the cells were treated with CD206 antibody and applied for flow cytometry. Flow cytometry results showing that M2 biomarker CD206 expression was downregulated in M2 phenotype cells treated with STING-NV for 24 h.

FIG. 14. RAW264.7 macrophages were cultured in LPS plus IFN-γ for 24 hours to induce an M1-like phenotype and IL-4 to induce an M2-like phenotype. UT M ϕ, untreated control. On day 2, M2 cells were treated with 1.5 μg/mL CDG delivered as free CDG, F-CDG, 5C-NPs, STING-NVs or Lipo-CDG. On day 3, the cells were treated with CD86 antibody and applied for flow cytometry. Flow cytometry results showing that STING-NV elevated the expression of M1 biomarker CD86 in M2 phenotype cells after 24 h.

FIG. 15. RAW264.7 macrophages were cultured in LPS plus IFN-γ for 24 hours to induce an M1-like phenotype and IL-4 to induce an M2-like phenotype. UT M ϕ, untreated macrophage control. On day 2, M2 cells were treated with 1.5 μg/mL CDG delivered as free CDG, F-CDG, 5C-NPs, STING-NVs or Lipo-CDG. On day 3, the cells were treated with MHC class UI (TA/TE) antibody and applied for flow cytometry. Flow cytometry results showing that STING-NVs elevated the expression of M1 biomarker MHC class II in M2 phenotype cells after 24 h.

FIG. 16A-D. STING-NVs mediated potent immune stimulation in DCs. (a-c) STING-NVs elevated the production of proinflammatory factors IL-6 (a), IL-12 (b), and IFN-α (c) in DCs. DC2.4 cells were treated with 5C-NPs, CDG, F-CDG or STING-NVs (1 μg/mL CDG equivalents) for 24 h. ELISA results suggest STING-NVs induced DC2.4 cells to secrete significantly more IFN-α, IL-6, and IL-12p40 than CDG or even F-CDG, after 24 h. (d) STING-NVs elevated the expression of co-stimulatory factors and MHC molecules in DCs. DC2.4 cells were treated with 5C-NP, soluble CDG, F-CDG or STING-NVs (1 μg/mL CDG equivalents). After 24 h, the cells were treated with CD80, CD86, or MHC class II (IA/IE) antibodies and applied for flow cytometry. Flow cytometry results showing that STING-NVs elevated the expression of co-stimulatory and MHC molecules in DC2.4 cells after 24 h. Data represent mean±SD. ****p<0.0001, by one-way ANOVA with a Tukey's post hoc test.

FIG. 17. Individual tumor growth curves in the immunotherapy studies of STING-NVs in B16F10 melanoma.

FIG. 18. Schematic illustration of pH-responsive STING-NVs that efficiently load CDG at physiological pH, stabilized CDG, delivered CDG to immune cells, conditionally release CDG in the acidic endosome, and facilitated endosome escape of CDG for cancer immunotherapy. CDG was loaded in DNA i-motif-coated PEG-b-PLA NPs by hydrogen bonding (i.e., G:C base pairing) under physiological pH (linear i-motifs). Under acidic conditions such as immune cell endosome, i-motifs form C-quadruplexes through protonated C:C+ base-pair formation, leading to dissociation of CDG from i-motifs and hence CDG release from STING-NVs. TBK1, TANK-binding kinase 1; IRF3, interferon regulatory factor 3; IKK, IκB kinase.

### DETAILED DESCRIPTION

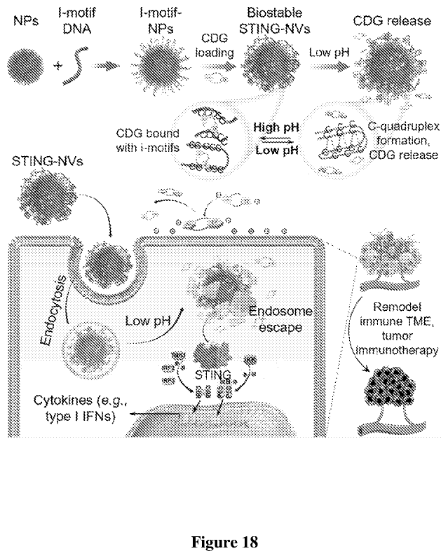

The present disclosure provides novel STING-NVs (nano-vaccines) that stimulate the immune system via CDN activation of the STING protein. The NVs are used, e.g. for treating cancer and viral infections. The NVs advantageously protect CDNs from enzymatic degradation, promote CDN delivery, enable conditional CDN release in the acidic endosome, and facilitate endosome escape of CDN in immune cells to remodel the tumor immune microenvironment for cancer immunotherapy, as well as for the treatment of viral infections. Specifically, as described in the Examples below, an exemplary CDN, CDG, was loaded into i-motif DNA nanoparticles to form STING-NVs under physiological pH via hydrogen bonding (i.e., G:C base pairing) between the guanosine (G) in CDG and the C in i-motif DNA that was coated on the polymer nanoparticles (NPs). When delivered into the acidic endosome of immune cells, CDG was released (dissociated) from the STING-NVs in response to the acidic pH and modulated the tumor immune microenvironment for cancer immunotherapy (FIG. 18). Notably, in in vivo experiments, administration of the STING-NVs caused no detectable side effects.

I-motifs are DNA with multiple domains of consecutive C bases. I-motifs display pH-responsive conformational reconfiguration between 1) linear strands under neutral or physiological pH, and 2) intermolecular or intramolecular four-stranded C-quadruplexes that are formed via protonated C:C+ base pairing under a dynamic pH range of 5-7.^{[12]} Taking advantage of this feature of i-motifs, we modified i-motif DNA on poly(D,L-lactide)-block-poly(ethylene glycol) (PEG-b-PLA) NPs to enable 1) CDG loading by binding with i-motifs at physiological pH, and 2) conditional CDG release upon the formation of C-quadruplexes under acidic conditions that competed with CDG for C:C+ base pairing. We optimized the lengths of i-motifs for CDG loading into NPs and validated conditional CDG release from the resulting STING-NVs under a range of acidic conditions. STING-NVs protected CDG from enzymatic degradation. STING-NVs were efficiently delivered into immune cells, and facilitated the endosome escape of CDG into cytosol where CDG can interact with STING. STING-NVs dramatically potentiated the immunostimulatory efficacy of CDG in both murine and human immune cells. STING-NVs elicited potent antitumor immune responses in vitro and in vivo, via, for example, repolarizing immunosuppressive M2-like macrophages to antitumor M1-like macrophages. Consequently, in a poorly immunogenic melanoma mouse model, STING-NVs showed superior immunotherapeutic efficacy relative to free CDG, fluoride-modified CDG (F-CDG) which is a current gold standard of biostable CDG, and liposomal CDG (Lipo-CDG).

In some aspects, the present disclosure provides a STING-NV which comprises at least three components: 1) a nano- or micro-particulate scaffold; 2) at least one type of i-motif DNA on the nano- or micro-particulate scaffold; and 3) at least one type of CDN attached to the i-motif DNA. Aspects of each component are discussed below. The term “STING-NV” is intended to encompass particles with both nanometer and micrometer dimensions.

### Nano- or Micro-Particulate Scaffold

The core of the STING-NVs disclosed herein is a nano- or micro-particulate scaffold, which may be referred to herein as a “scaffold” or “scaffolding particle” or “particle”. Generally, the scaffolds are self-assembled and they may be micellular in nature. As is understood by those of skill in the art, “nano-particulate” particles typically range in size from about 1 to 100 nanometers (nm) in diameter, or up to about 500 nm or more whereas particles ranging in size from about 0.5 μm up to about 0.5 mm are considered “micro-particulate”.

The scaffolding particles of the NVs disclosed herein may be formed from any of a variety of ingredients, provided they are biocompatible and non-toxic. In some cases, when assembled into a particle, the particle may be, but is not always, micellular. General requirements for the scaffolding particles are that, once assembled, they must be amenable to (capable of) being modified by the physical or chemical attachment of/attachment to i-motif DNA. Thus, functional chemical groups or inorganic elements (e.g., gold) that can be used for covalent or covalent attachment of i-motif may be present on the scaffolding particles in order to bind to the corresponding reactive groups on the i-motif DNA.

Examples of nano- or micro-particles include but are not limited to: quantum dots, carbon nanotubes, nanoshells, dendrimers, liposomes, lipid nanoparticles, metal nanoparticles (such as gold nanoparticles), quantum dot nanoparticles, magnetic nanoparticles, carbon nanoparticles, latex beads/fluorescent nanoparticles, and cellulose nanoparticles. Exemplary biodegradable polymers used in the fabrication of polymeric nanoparticles include but are not limited to: poly(lactide) (PLA), poly(lactide-co-glycolide) (PLGA) copolymers, poly (ε-caprolactone) (PCL), and poly(amino acids) and also some natural polymers like alginate, chitosan, gelatin, and albumin. Other exemplary nanoparticles and microparticles that may be employed include but are not limited to those described in published US patent application 220105191, the complete contents of which are hereby incorporated by reference in entirety.

In some aspects, the material that is used is an amphiphilic diblock copolymer made from (and thus comprising) poly(ethylene glycol)-block-poly(D,L-lactic acid) (PEG-b-PLA), i.e. the scaffolding particle is a “PEG-b-PLA scaffolding particle”.

In some aspects, the scaffolding particles are made up of mixtures of molecules which are capable of self-assembly and which, once assembled, provide interior and exterior surface functional groups which permit i-motif DNA to attach. For example, about 2, 3, 4, 5, 6, 7, 8, 9 or 10 different types of molecules may be mixed.

Generally, after formation, the scaffolding particles have a hydrodynamic diameter of nanoparticulate scaffolding particles ranging from about 10 nm to about 50 μm, for example, from about 20 to 400 nm or from about 30 to 300 nm, or from about 40 to 200 nm, about 50 to 100 nm, such as about 50, 60, 70, 80, 90, or 100 nm. In some aspects, the hydrodynamic diameter is about 80 nm, such as about 78±35 nm.

### I-Motif DNA

i-motif DNAs, short for “intercalated-motif DNAs”, are cytosine-rich four-stranded quadruplex DNA structures. i-motifs comprise antiparallel tracts of oligodeoxynucleotides strands that contain mostly cytosine residues. The interactions occur by the hemi protonation of cytosine residues and non-Watson Crick base pairing, i.e. by Hoogsteen base pairing. There are two main intercalated topologies that i-motifs can be classified in: 3′-E, when the outmost C:C+ base pair is at the 3′-end, and 5′-E, where the outermost C:C+ base pair is at the 5′-end. When comparing the two topologies, the 3′-E topology is more stable due to increased sugar-sugar contacts. The interactions of the sugar-sugar contacts along the narrow grooves allows for optimal backbone twisting, which ultimately contributes to formation of stacking bases and the stability of the molecule. However, the overall stability of i-motif structures is dependent on the number of cytosine residues that are interacting with each other. This means that as more cytosine residues interact through hydrogen bonding, the more stable the molecule will be. Other factors that affect the stability of the molecules include temperature, salt concentration and pH of the environment. Some i-motif complexes are most stable at a slightly acidic pH (e.g. between 4.2 and 5.2).

The i-motif DNA used in the present disclosure may be naturally occurring or synthetic, with synthetic forms being preferred.

As described herein, to form the present STING-NVs, the i-motif DNA must be amenable to/capable of binding to a scaffolding particle as described herein, by covalent or non-covalent attachment of the i-motif DNA to the scaffolding particle, in which covalent chemical bonding, ionic bonding, and/or ionic bonding and van der Waals bonding are encompassed. However the binding occurs, the resulting particle-i-motif DNA complex must be stable at a wide range of pH values so that the complex can i) initially stably carry CDNs into environments with pH values close to or higher than physiological pH (e.g. about 7.2-about 7.5), and ii) yet remain stable at more acidic pH (e.g. below 7.0, such as between 5 and 7, in order to effect release of the bound CDN from the scaffolding particle-i-motif complex within endosomes, without dissociating itself, i.e. the particle-i-motif DNA complex generally remains intact even though the CDN is released. Early endosomes maintain pH at about 6.5, while late endosomes are at about pH 5.5.

Binding of the i-motif DNA to a scaffolding particle may be carried out by any of several chemical or physical approaches that result in covalent or noncovalent attachment of i-motif DNA with the scaffold. For example, one or the other or both of the two reactants can be chemically modified by the attachment of a reactive functional group so that, when the two are combined, the i-motif DNA is attached to the scaffolding particle. Examples of reactive groups which can be used for such modifications include but are not limited to reactive thiol groups, amine, carboxyl, azide, alkyne, maleimide, and hydroxyl; Examples of noncovalent bonding can be introduced from hydrogen binding, van der Waals interaction, hydrophobic interactions, ionic binding, and the like.

In some aspects, a thiol group is used to modify the i-motif DNA.

In some aspects, only one type of i-motif-DNA is bound to a scaffolding particle. In other aspects, a mixture of two of more types of i-motif-DNAs are bound to a scaffolding particle, such as from about 2, 3, 4, 5, 6, 7, 8, 9 or 10 different types.

Accordingly, in addition to binding to the scaffolding particle, the i-motif DNA must be amenable to/capable of binding to one or more types of CDNs, generally via ionic bonding. The attachment of CDNs to an i-motif-modified scaffolding particle (i.e. the loading of the i-motif-modified scaffolding particle with CDNs) typically occurs when positively charged CDNs replace positively charged cytosine bases in the i-motifs at the scaffolding particles. Under acidic conditions, C bases of the i-motif DNA compete with CDN for C:C+ base pairing, and the C:C+ base pairing attaches the CDN to the i-motif-particle complex.

In the present STING-NVs, the type of i-motif DNA that is used may be either 3′E or 5′E. As understood in the art, i-motif DNA is cytosine-rich and comprises a plurality of cytosine-rich domains. For example, from about 2 to about 100 cytosine-rich domains may be present, such as about 2, 3, 4, 5, 6, 7, 8, 9, 10 . . . 15 . . . 20 . . . 30 . . . 40 . . . 50 . . . 60 . . . 70 . . . 80 . . . 90 . . . to about 100 or more, where “ . . . ” indicates all whole integers between each two numbers. Each cytosine rich domain contains from about 2-50 adjacent, consecutive cytosines. (e.g. at least about 2 or more, and usually about 3 or more, and up to, for example 50 or more, such as about 2, 3, 4, 5, 6, 7, 8, 9, 10 . . . 15 . . . 20 . . . 30 . . . 40 . . . to about 50 or more, where “ . . . ” indicates all whole integers between each two numbers. In some aspects, the cytosine-rich i-motif DNA comprises from 1-10 cytosine-rich domains, such as 1, 2, 3, 4, 5, 6, 7, 8, 9 or 10 cytosine-rich domains, interrupted (segregated) by about 1-3 non-cytosine bases. In some aspects, the number of cytosine-rich domains is four. In some aspects, each cytosine-rich domain comprises at least 2 to about 10 consecutive cytosines, such as about 2, 3, 4, 5, 6, 7, 8, 9 or 10 consecutive cytosines. In some aspects, each cytosine-rich domain comprises at least 3 consecutive cytosines, such as 3, 4, 5, 6, or 7. In some aspects, a cytosine-rich domain comprises 5 consecutive cytosines.

The remainder of the i-motif DNA is generally made up of other bases, e.g. thymine, adenine, guanine, that are interspersed between cytosine-rich domains.

In some aspects, alternative bases may be incorporated into the i-motif DNA, e.g. to adjust (increase or decrease) stability. For example, cytosine may be replaced with 5-methylcytosine and thymine may be replaced with 5-propynyl uracil. Both of these substitutions increase the stability of the i-motif structure. These and other modifications of the bases may be used to modulate the pH/temperature-dependent folding patterns of i-motifs. Other modifications can include but are not limited to phosphorothioate, 5′-F, 5′-methyl, 5′-O-methyl.

i-motif DNAs that are suitable for use in the present STING-NVs generally comprise from about 10 to about 500 bases, with no theoretical upper limit. In some aspects, the number of bases is about 10, 15, 20, 25, 30, 35, 40, 45, 50, 55, 60, 65, 70, 75, 80, 85, 90, 95, 100, 150, 200, 250, 300, 350, 400, 450 or 500. More usually, about 10 to 50 or so bases are present, such as ore from about 25 to about 40 bases.

The ratio of scaffolding particle material to i-motif in the NVs is generally from about 75:1 to about 1:1, such as about 75:1, 74:1, 73:1, 72:1, 7.1:1, 70:1 . . . to about 5:1, 4:1, 3:1, 2:1 or 1:1, including all integers between represented by “ . . . ”.

### Cyclic Dinucleotides

The third component in the STING-NVs disclosed herein is a cyclic dinucleotide (CDN), particularly cyclic dimeric guanosine monophosphate (c-di-GMP), which interacts with polyC (i-motif) with relatively strong affinity.

The CDNs that are used in the NVs, for example one or more of cyclic dimeric guanosine monophosphate (CDG), cyclic dimeric adenosine monophosphate (CDA), and cyclic GMP-AMP (cGAMP) are agonists of STING, that is they bind to STING and cause its activation, i.e. they function as STING agonists when present in the cell and when in contact with STING. The present NVs serve to increase the local concentration of CDNs in cells, e.g. in cells of a subject, and especially in the vicinity of STING proteins, so as to activate STING to a level that is higher than that which would otherwise (but for the use of the NVs) be naturally present in a cell. The result is STING activation and the benefits that follow, namely stimulation of the immune system of a subject to whom the STING-NVs are administered.

Typically, the CDN loading capacity and loading efficiency are calculated by the following equations:

Loading capacity=(CDN weight in scaffold particles)/(weight of scaffold particles);

Loading efficiency=(CDN weight in scaffold particles)/(total weight of CDN).

For the STING-NVs disclosed herein, the CDN loading capacity is generally from about 0.5 to about 5%, such as about 0.5, 1.0, 1.5, 2.0, 2.5, 3.0, 3.5, 4.0, 4.5 or 5.0% w/w although in some cases, the loading may be higher, e.g. up to about 10%.

For the STING-NVs disclosed herein, the CDN loading efficiency is generally from about 10 to about 100%, such as about 10, 20, 30, 40, 50, 60, 70, 80, 90 or 100% w/w. In some aspects, the loading efficiency is about 50%.

The ratio of CDN to the number of consecutive cytosines in i-motif-scaffolding particle complex ranges from about 1:2 to about 1:100, such as about 1:2, 1:2.1 . . . or 1:100 w/w, including all 0.1 decimal integers between represented by “ . . . ”.

The loading content of CDN per i-motif-scaffolding particle complex is generally in the range of from about 0.5 to about 50.0%, such as about 0.5, 1.0, 1.5, 2.0, 2.5, 3.0, 3.5, 4.0, 4.5, 5.0 . . . 10 . . . 20 . . . 30 . . . 40 . . . or 50% w/w, including all integers to one decimal point between, as represented by “ . . . ”.

Further Optional Modifications of the NVs and/or of the Components Thereof

In some aspects, one or more components of the NVs may be modified by attachment of one or more ligands, for example, detectable labels and/or therapeutic agents, and/or targeting ligands, antigen molecules, other immunostimulatory agents, imaging or signaling probes, etc. Such modifications may be introduced before and/or after the i-motif DNA is bound to a scaffolding particle, and/or before and/or after the CDN is bound to the i-motif DNA that is already bound to the scaffolding particle.

Modifications may be made to any of the three components of the STING-NVs: the scaffolding particle, the i-motif DNA or the CDN. Any component of the NVs can be modified by attachment of a ligand, such as a detectable label, a therapeutic agent, etc. as long as the ligand, label, etc. does not interfere with the intended function of the NVs (release of the CDN under acidic conditions).

In some aspects, the ligand is a detectable label. Examples of suitable detectable labels include but are not limited to: a magnetic label, a fluorescent moiety, an enzyme, a chemiluminescent probe, a metal particle, a non-metal colloidal particle, a polymeric dye particle, a pigment molecule, a pigment particle, an electrochemically active species, semiconductor nanocrystal or other nanoparticles including quantum dots or gold particles, fluorophores, quantum dots, or radioactive labels. Protein labels include green fluorescent protein (GFP) and variants thereof (e.g., cyan fluorescent protein and yellow fluorescent protein); and luminescent proteins such as luciferase, as described below. Radioactive labels include without limitation radioisotopes (radionuclides), such as ³H, ¹¹C, ¹⁴C, ¹⁸F, ³²P, ³⁵S, ⁶⁴Cu, ⁶⁸Ga, ⁸⁶Y, ⁹⁹Tc, ¹¹¹In, ¹²³I, ¹²⁴I, ¹²⁵I, ¹³¹I, ¹³³Xe, ¹⁷⁷Lu, ²¹¹At, or ²¹³Bi. Generally, the labels are fluorescent labels which include without limitation a rare earth chelate (e.g., europium chelate), rhodamine; fluorescein types including without limitation FITC, 5-carboxyfluorescein, 6-carboxy fluorescein; a rhodamine type including without limitation TAMRA; dansyl; Lissamine; cyanines; phycoerythrins; Texas Red; Cy3, Cy5, dapoxyl, NBD, Cascade Yellow, dansyl, PyMPO, pyrene, 7-diethylaminocoumarin-3-carboxylic acid and other coumarin derivatives, Marina Blue™, Pacific Blue™, Cascade Blue™, 2-anthracenesulfonyl, PyMPO, 3,4,9,10-perylene-tetracarboxylic acid, 2,7-difluorofluorescein (Oregon Green™488-X), 5-carboxyfluorescein, Texas Red™-X, Alexa Fluor 430, 5-carboxytetramethylrhodamine (5-TAMRA), 6-carboxytetramethylrhodamine (6-TAMRA), BODIPY FL, bimane, and Alexa Fluor 350, 405, 488, 500, 514, 532, 546, 555, 568, 594, 610, 633, 647, 660, 680, 700, and 750, and derivatives thereof, among many others. See, e.g., “The Handbook-A Guide to Fluorescent Probes and Labeling Technologies,” Tenth Edition, available on the internet at probes (dot) invitrogen (dot) corn/handbook. The fluorescent label can be one or more of FAM, dRHO, 5-FAM, 6FAM, dR6G, JOE, HEX, VIC, TET, dTAMRA, TAMRA, NED, dROX, PET, BHQ, Gold540 and LIZ.

Examples of ligands that are especially appropriate for the i-motif DNA component of the NVs include e.g. fluorescent probes, examples of which include but are not limited to: thiazole orange, 2,2′-diethyl-9-methylselenacarbocyanine bromide (DMSB), crystal violet, berberine neutral red, thioflavin T, and perylene tetracarboxylic acid diimide derivative (PTCDI). In addition, radioactive bases may be used to form the i-motif DNA and render it detectable. Other examples of modifications of the i-motif DNA include but are not limited to: colormetric dyes (e.g. methylene blue), various metals (e.g. gold), radioisotopes, magnetic sensitive probes (e.g., iron oxide); targeting ligands such as antibodies, peptide ligands, aptamers, small molecular ligands such as mannose, and the like.

In further aspects, one or more therapeutic agents may also be attached to the NVs via one or more of the components thereof. Examples of such agents include those listed below as agents that can be administered with the NVs, for example, anti-cancer agents.

### Pharmaceutical Compositions

The compounds described herein (e.g. STING-NVs) are generally delivered (administered) as a pharmaceutical or therapeutic composition. Such pharmaceutical compositions generally comprise at least one of the disclosed therapeutic agents that is substantially purified, i.e. one or more than one (a plurality) of different NVs (e.g. 2 or more such as 2, 3, 4, 5, 6, 7, 8, 9, 10 or more) may be included in a single formulation. Accordingly, the present invention encompasses such formulations/compositions. The compositions generally include one or more substantially purified compounds as described herein, and a pharmacologically suitable (physiologically compatible) carrier. In some aspects, such compositions are prepared as liquid solutions or suspensions, or as solid forms such as tablets, pills, powders and the like. Solid forms suitable for solution in, or suspension in, liquids prior to administration are also contemplated (e.g. lyophilized forms of the compounds), as are emulsified preparations. In some aspects, the liquid formulations are aqueous or oil-based suspensions or solutions. In some aspects, the active ingredients are mixed with excipients which are pharmaceutically acceptable and compatible with the active ingredients, e.g. pharmaceutically acceptable salts. Suitable excipients include, for example, water, saline, dextrose, glycerol, ethanol and the like, or combinations thereof. In addition, a composition may contain minor amounts of auxiliary substances such as wetting and/or emulsifying agents, pH buffering agents, preservatives, and the like. In some aspects, it is desired to administer an oral form of the composition so various thickeners, flavorings, diluents, emulsifiers, dispersing aids or binders and the like are added. The compositions of the present invention may contain any such additional ingredients so as to provide the composition in a form suitable for administration. The final amount of compound in the formulations varies but is generally from about 1-99%. Still other suitable formulations for use in the present invention are found, for example in Remington's Pharmaceutical Sciences, 22nd ed. (2012; eds. Allen, Adejarem Desselle and Felton).

Some examples of materials which can serve as pharmaceutically acceptable carriers include, but are not limited to, ion exchangers, alumina, aluminum stearate, lecithin, serum proteins (such as human serum albumin), buffer substances (such as Tween® 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions. In addition, other non-toxic compatible lubricants such as sodium lauryl sulfate and magnesium stearate, as well as coloring agents, releasing agents, coating agents, sweetening, flavoring and perfuming agents, preservatives and antioxidants may also be present in the composition, according to the judgment of the formulator.

“Pharmaceutically acceptable salts” refers to the relatively non-toxic, inorganic and organic acid addition salts, and base addition salts, of compounds of the present invention. These salts can be prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulfamates, malonates, salicylates, propionates, methylene-bis-.beta.-hydroxynaphthoates, gentisates, isethionates, di-p-toluoyltartrates, methanesulfonates, ethanesulfonates, benzenesulfonates, p-toluenesulfonates, cyclohexylsulfamates and laurylsulfonate salts, and the like. See, for example S. M. Berge, et al., “Pharmaceutical Salts,” J. Pharm. Sci., 66, 1-19 (1977) which is incorporated herein by reference. Base addition salts can also be prepared by separately reacting the purified compound in its acid form with a suitable organic or inorganic base and isolating the salt thus formed. Base addition salts include pharmaceutically acceptable metal and amine salts. Suitable metal salts include the sodium, potassium, calcium, barium, zinc, magnesium, and aluminum salts. The sodium and potassium salts are generally preferred. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which have sufficient basicity to form a stable salt, and preferably include those amines which are frequently used in medicinal chemistry because of their low toxicity and acceptability for medical use, examples of which include but are not limited to: ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, and dicyclohexylamine, and the like.

### Administration

The NVs of the invention may be administered in the form of a pharmaceutical composition, as defined below. Preferably, the NVs are administered in a therapeutically effective amount. By a “therapeutically effective amount” is meant a sufficient amount of the agent to treat at least one symptom of a disease or condition at a reasonable benefit/risk ratio applicable to any medical treatment. In particular, the effect of the STING-NVs disclosed herein is stimulation of the immune system, e.g. of a cancer patient and/or a patient with a viral infection. Successful stimulation of the immune system results in the increase in various immune system markers and overall in an increase in the patient's ability to overcome cancer or the viral infection, and/or an increase in the effectiveness of one or more anticancer or antiviral drugs that are administered to the patient with the STING-NVs. Thus, a decrease in cancer or viral associated symptoms is then observed.

A “therapeutically effective amount of the NVs” is an amount that induces, ameliorates or causes an improvement in the pathological symptoms, disease progression, or physical conditions associated with the disease affecting the patient. The present methods find use in treating, i.e., reducing, relieving, alleviating, ameliorating, or inhibiting symptoms of disease in a patient in need thereof. The terms “reduce”, “inhibit”, “relieve”, “alleviate” refer to the detectable decrease in symptoms of the disease, as determined by a trained clinical observer. A reduction in symptoms can be measured by self-assessment (e.g., by reporting of the patient), by applying various measurement assays well known in the art (e.g., medical imaging, blood tests to detect antigens, markers of inflammation and/or cancer cells, viral load, tumor shrinkage, etc.). Determination of a reduction of one or more symptoms can be made by comparing patient status before and after treatment. In some aspects, disease symptoms may be eliminated, i.e. the patient may be fully cured.

The pharmaceutical compositions are administered in vivo by any suitable route including but not limited to: inoculation or injection (e.g. intravenous, intraperitoneal, intramuscular, subcutaneous, intra-aural, intra-articular, intra-mammary, and the like); intralesionally; topical application (e.g. on areas such as eyes, skin, in ears; by absorption through epithelial or mucocutaneous linings (e.g., nasal, oral, vaginal, rectal, gastrointestinal mucosa, and the like); and/or by inhalation (e.g. as a mist or spray). Formulations suitable for a particular mode of administration are also encompassed, e.g. liquids for intravenous administration; pills, capsules, liquids, etc. for oral administration; creams, ointments and suppositories for intravaginal or rectal administration; as eye drops for administration to the eye; etc. In preferred aspects, delivery is intralesional.

Subjects to whom the pharmaceutical compositions are administered include but are not limited to mammals. In some aspects, the mammal is a human. However, veterinary uses are also encompassed, such as for the treatment of companion pets (cats, dogs, rodents, rabbits, etc.), animals in zoos or preserves, animals raised commercially, especially those used for breeding such as prize horses or cattle, etc.

The frequency of administration is best determined by a medical professional, but will generally be from about 1-4 times per day, or once per day (daily), or every other day, or 1-7 times per week, or weekly, every two weeks, every third week, monthly, every 2-11 months, yearly, etc. In some aspects, administrations may be given on a regular basis throughout the lifetime of the patient, e.g. monthly, every 3 or 6 or 9 months, yearly, every 5 or 10 years, etc. as needed. This is especially beneficial for subjects who are chronically immune compromised.

The amount of STING-NV that is administered in a single dose generally ranges from about 0.001 to about 100 mg CDG per recipient patient. Those of skill in the art will recognize that the amounts and frequencies, and even the mode of administration, may vary depending on any of several factors, e.g. age, general health, gender, weight, etc. and are best determined by a medical professional in light of clinical trial data.

The compositions may be administered in conjunction with other treatment modalities such as but not limited to: substances that boost the immune system, various chemotherapeutic agents, pain medication, surgery, radiation therapy, and the like.

In particular, the NVs may be administered with one or more anti-cancer agents, examples of which include but are not limited to: alkylating agents, such as mustard gas derivatives (Mechlorethamine, Cyclophosphamide (Cytoxan), Chlorambucil (Leukeran), Melphalan, and Ifosfamide), ethylenimines (Thiotepa (Thioplex) and Hexamethylmelamine), alkylsulfonates (Busulfan (Myleran)), hydrazines and triazines (Altretamine (Hexalen), Procarbazine (Matulane), Dacarbazine (DTIC) and Temozolomide), nitrosureas (Carmustine, Lomustine and Streptozocin), and metal salts (Carboplatin, Cisplatin (Platinol), and Oxaliplatin), Mechlorethamine, and Melphalan (Alkeran); plant alkaloids, terpenoids and topoisomerase inhibitors, such as vinca alkaloids (Vincristine (Oncovin), Vinblastine (Velban), Vindesine, and Vinorelbine), taxanes (Paclitaxel (Taxol) and Docetaxel (Taxotere)), podophyllotoxins (Etoposide and Tenisopide), and camptothecan analogs (Irinotecan and Topotecan); antitumor antibiotics, such as anthracyclines (Doxorubicin (Adriamycin, Rubex, Doxil), Daunorubicin, Epirubicin, Mitoxantrone, Idarubicin, Duocarmycin, and Dactinomycin (Cosmegen)), chromomycins (Dactinomycin and Plicamycin (Mithramycin)), and miscellaneous (Mitomycin and Bleomycin (Blenoxane));
antimetabolites, such as folic acid antagonists (Methotrexate), pyrimidine antagonists (5-Fluorouracil, Foxuridine, Cytarabine, Flurouracil (5-FU), Capecitabine, and Gemcitabine), purine antagonists (6-Mercaptopurine (Purinethol) and 6-Thioguanine), 6-Thiopurines, and adenosine deaminase inhibitor (Cladribine (Leustatin), Fludarabine, Nelarabine and Pentostatin), Azacitidine, Thioguanine, and Cytarabine (ara-C); topoisomerase Inhibitors, such as topoisomerase I inhibitors (Ironotecan, topotecan), and topoisomerase II inhibitors (Amsacrine, etoposide, etoposide phosphate, teniposide); hormonal agents, exemplified by Estrogen and Androgen Inhibitors (Tamoxifen and Flutamide), Gonadotropin-Releasing Hormone Agonists (Leuprolide and Goserelin (Zoladex)), Aromatase Inhibitors (Aminoglutethimide and Anastrozole (Arimidex)); DNA hypomethylating agents, e.g., Azacitidine, Decitabine; Poly(adenosine diphosphate [ADP]-ribose) polymerase (PARP) pathway inhibitors, such as Iniparib, Olaparib, Veliparib; PI3K/Akt/mTOR pathway inhibitors, e.g., Everolimus; Histone deacetylase (HDAC) inhibitors, e.g., Vorinostat, Entinostat (SNDX-275), Mocetinostat (MGCD0103), Panobinostat (LBH589), Romidepsin, Valproic acid; Cyclin-dependent kinase (CDK) inhibitors, e.g., Flavopiridol, Olomoucine, Roscovitine, Kenpaullone, AG-024322 (Pfizer), Fascaplysin, Ryuvidine, Purvalanol A, NU2058, BML-259, SU 9516, PD-0332991, P276-00; Heat shock protein (HSP90) inhibitors, e.g., Geldanamycin, Tanespimycin, Alvespimycin, Radicicol, Deguelin, and BIIB021; Murine double minute 2 (MDM2) inhibitors, e.g., Cis-imidazoline, Benzodiazepinedione, Spiro-oxindoles, Isoquinolinone, Thiophene, 5-Deazaflavin, Tryptamine; Anaplastic lymphoma kinase (ALK) inhibitors, e.g., Aminopyridine, Diaminopyrimidine, Pyridoisoquinoline, Pyrrolopyrazole, Indolocarbazole, Pyrrolopyrimidine, Dianilinopyrimidine; Poly [ADPribose] polymerase (PARP) inhibitors, illustrated by Benzamide, Phthalazinone, Tricyclic indole, Benzimidazole, Indazole, Pyrrolocarbazole, Phthalazinone, Isoindolinone; and miscellaneous anticancer drugs, exemplified by Amsacrine, Asparaginase (El-spar), Hydroxyurea, Mitoxantrone (Novantrone), Mitotane (Lysodren), Maytansinoid, Retinoic acid Derivatives, Bone Marrow Growth Factors (sargramostim and filgrastim), Amifostine, agents disrupting folate metabolism, e.g., Pemetrexed, ribonucleotide reductase inhibitors (Hydroxyurea), adrenocortical steroid inhibitors (Mitotane), enzymes (Asparaginase and Pegaspargase), antimicrotubule agents (Estramustine), and retinoids (Bexarotene, Isotretinoin, Tretinoin (ATRA)).

Chemotherapy drugs that are illustrative of the small molecule drug subcategory are Actinomycin-D, Alkeran, Ara-C, Anastrozole, BiCNU, Bicalutamide, Bleomycin, Busulfan, Capecitabine, Carboplatin, Carboplatinum, Carmustine, CCNU, Chlorambucil, Cisplatin, Cladribine, CPT-11, Cyclophosphamide, Cytarabine, Cytosine arabinoside, Cytoxan, Dacarbazine, Dactinomycin, Daunorubicin, Dexrazoxane, Docetaxel, Doxorubicin, DTIC, Epirubicin, Ethyleneimine, Etoposide, Floxuridine, Fludarabine, Fluorouracil, Flutamide, Fotemustine, Gemcitabine, Hexamethylamine, Hydroxyurea, Idarubicin, Ifosfamide, Irinotecan, Lomustine, Mechlorethamine, Melphalan, Mercaptopurine, Methotrexate, Mitomycin, Mitotane, Mitoxantrone, Oxaliplatin, Paclitaxel, Pamidronate, Pentostatin, Plicamycin, Procarbazine, Steroids, Streptozocin, STI-571, Streptozocin, Tamoxifen, Temozolomide, Teniposide, Tetrazine, Thioguanine, Thiotepa, Tomudex, Topotecan, Treosulphan, Trimetrexate, Vinblastine, Vincristine, Vindesine, Vinorelbine, VP-16, and Xeloda.

The subcategory of biologic chemotherapy drugs includes, without limitation, Asparaginase, AIN-457, Bapineuzumab, Belimumab, Brentuximab, Briakinumab, Canakinumab, Cetuximab, Dalotuzumab, Denosumab, Epratuzumab, Estafenatox, Farletuzumab, Figitumumab, Galiximab, Gemtuzumab, Girentuximab (WX-G250), Herceptin, Ibritumomab, Inotuzumab, Ipilimumab, Mepolizumab, Muromonab-CD3, Naptumomab, Necitumumab, Nimotuzumab, Ocrelizumab, Ofatumumab, Otelixizumab, Ozogamicin, Pagibaximab, Panitumumab, Pertuzumab, Ramucirumab, Reslizumab, Rituximab, REGN88, Solanezumab, Tanezumab, Teplizumab, Tiuxetan, Tositumomab, Trastuzumab, Tremelimumab, Vedolizumab, Zalutumumab, and Zanolimumab.

Alternatively, if the patient is treated for a viral infection, the NVs may be administered with one or more antiviral agents including but not limited to: an anti-viral vaccine, anti-viral antibodies, immunoglobulins, an antiviral drug such as ribavirin, interferon, nucleoside analogs (e.g. zidovudine, acyclovir, acyclovir prodrugs, famciclovir, gangcyclovir, vidarabine, idoxuridine, trifluridine, famcyclovir, valacyclovir and ribavirin), n-docosanoll foscarnet, amantadine, rimantadine, saquinavir, indinavir, ritonavir, idoxuridine, amantadine, alpha-interferons and other interferons, AZT, sialidase inhibitors, protease inhibitors and combinations thereof.

One or more types of STING-NVs may be administered with one or more of the listed anti-cancer agents. By “with” is meant they may be administered as a single composition, or as separate compositions, where the administration is simultaneous, sequential or at various timed intervals suitable for each drug.

Diseases and Conditions that are Treated

In some aspects, the STING-NVs are used in the treatment of cancer, especially to stimulate the immune system of a cancer patient to fight off cancer cells/tumor cells. Examples of cancers that are so treated include but are not limited to:
Acute Lymphoblastic Leukemia (ALL), Acute Myeloid Leukemia (AML), Adrenocortical Carcinoma, AIDS-Related cancers, Kaposi Sarcoma, AIDS-Related Lymphoma, Primary CNS Lymphoma, Anal cancer, Appendix cancer, Astrocytomas, Atypical Teratoid/Rhabdoid Tumor, Central Nervous System, Basal Cell Carcinoma, Bile Duct cancer, Bladder cancer, Bone cancer, Ewing Sarcoma Family of Tumors, Osteosarcoma and Malignant Fibrous Histiocytoma, Brain Stem Glioma, Brain Tumor (e.g. Astrocytomas, Brain and Spinal Cord Tumors, Brain Stem Glioma, Central Nervous System Atypical Teratoid/Rhabdoid Tumor, Central Nervous System Embryonal Tumors, Central Nervous System Germ Cell Tumors, Craniopharyngioma, Ependymoma), Breast cancer, Bronchial Tumors, Burkitt Lymphoma, Carcinoid Tumor, Gastrointestinal, Cardiac (Heart) Tumors, Central Nervous System (e.g. Atypical Teratoid/Rhabdoid Tumors, Embryonal Tumors, Germ Cell Tumors, Lymphomas), Cervical cancer, Childhood cancers, Cholangiocarcinoma, Chordoma, Chronic Lymphocytic Leukemia (CLL), Chronic Myelogenous Leukemia (CML), Chronic Myeloproliferative Neoplasms, Colon cancer, Colorectal cancer, Craniopharyngioma, Cutaneous T-Cell Lymphoma, Ductal Carcinoma In Situ (DCIS), Embryonal Tumors, Endometrial cancer, Ependymoma, Esophageal cancer, Esthesioneuroblastoma, Ewing Sarcoma, Extracranial Germ Cell Tumor, Extragonadal Germ Cell Tumor, Eye cancer, Intraocular Melanoma, Retinoblastoma, Fallopian Tube cancer, Fibrous Histiocytoma of Bone, Malignant, and Osteosarcoma, Gallbladder v, Gastric (Stomach) v, Gastrointestinal Carcinoid Tumor, Gastrointestinal Stromal Tumors (GIST), Hairy Cell Leukemia, Head and Neck v, Heart cancer, Hepatocellular (Liver) cancer, Histiocytosis, Langerhans Cell, Hodgkin Lymphom, Hypopharyngeal cancer, Intraocular Melanoma, Islet Cell Tumors, Pancreatic Neuroendocrine Tumors, Kaposi Sarcoma, Kidney (Renal Cell, Wilms Tumor and Other Childhood Kidney Tumors), Langerhans Cell Histiocytosis, Laryngeal cancer, Leukemia (Acute Lymphoblastic (ALL), Acute Myeloid (AML), Chronic Lymphocytic (CLL), Chronic Myelogenous (CML), Hairy Cell), Lip and Oral Cavity cancer, Liver cancer (Primary), Lung cancer (Non-Small Cell, Small Cell), Lymphoma, Macroglobulinemia, Hodgkin Lymphoma, Malignant Fibrous Histiocytoma of Bone and Osteosarcoma Melanoma, Intraocular (Eye), Merkel Cell Carcinoma, Mesothelioma, Malignant, Metastatic Squamous Neck cancer with Occult Primary Mouth cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia, Chronic (CML), Myeloid Leukemia, Acute (AML), Myeloma, Multiple, Myeloproliferative Neoplasms, Chronic, Nasal Cavity and Paranasal Sinus Cancer, Nasopharyngeal cancer, Neuroblastoma, Non-Hodgkin Lymphoma, Oral cancer, Oral Cavity cancer, Lip and Oropharyngeal cancer, Osteosarcoma and Malignant Fibrous Histiocytoma of Bone, Ovarian cancer, Epithelial, Germ Cell Tumor, Low Malignant Potential Tumor, Pancreatic cancer, Pancreatic Neuroendocrine Tumors (Islet Cell Tumors), Papillomatosis, Paraganglioma, Paranasal Sinus and Nasal Cavity cancer, Parathyroid cancer, Penile cancer, Pharyngeal cancer, Pheochromocytoma, Pituitary Tumor, Plasma Cell Neoplasm/Multiple Myeloma, Pleuropulmonary Blastoma, Primary Central Nervous System (CNS) Lymphoma, Primary Peritoneal cancer, Prostate cancer, Rectal cancer, Renal Cell (Kidney) cancer, Renal Pelvis and Ureter, Transitional Cell cancer, Retinoblastoma, Rhabdomyosarcoma, Salivary Gland cancer, Sarcoma (Ewing, Kaposi, Osteosarcoma, Rhabdomyosarcoma, Soft Tissue, Uterine), Sézary Syndrome, Skin cancer, Small Intestine cancer, Squamous Cell Carcinoma, Squamous Neck cancer, Metastatic Stomach (Gastric) cancer, T-Cell Lymphoma, Cutaneous, Testicular cancer, Throat cancer, Thymoma and Thymic Carcinoma, Thyroid Cancer, Ureter and Renal Pelvic cancer, Transitional Cell cancer, Urethral cancer, Uterine cancer, Vaginal cancer, vulvar cancer, Wilms Tumor, etc.

In other aspects, the NVs disclosed herein are used to treat subject having a viral infection. Examples of viruses which cause infections that can be treated using the NVs include but are not limited to: Adenoviridae, Coronaviridae, Filoviridae, Flaviviridae, Hepadnaviridae, Herpesviridae, Orthomyxoviridae, Paramyxovirinae, Pneumovirinae, Picomaviridae, Poxyiridae, Retroviridae, Togaviridae. Parainfluenza, Influenza, H5N1, Marburg, Ebola, Zika, RSV, HPV, HBV, HIV, Polio, Severe acute respiratory syndrome coronavirus (SARS-COV), Middle eastern respiratory syndrome coronavirus (MERS-COV), yellow fever virus, human respiratory syncytial, Hantavirus and Vaccinia virus. In some aspects, the virus is a coronavirus such as SARS coronavirus (SARS-CoV), which causes severe acute respiratory syndrome (SARS); MERS coronavirus (MERS-CoV), which causes Middle East respiratory syndrome (MERS); and SARS-CoV-2, which causes coronavirus disease 2019 (COVID-19).

In other aspects, the NVs disclosed herein are used as an immunomodulatory adjuvants of vaccines to improve the prophylactic efficacy of the said vaccines. Examples of vaccines include but are not limited to cancer therapeutic vaccines, cancer prophylactic vaccines, viral vaccines, bacterial and/or fungal vaccines, and anti-addiction vaccines.

### Methods of Making the Sting-NVs

Encompassed herein are methods of making STING-NVs as described herein. The methods comprise steps of: 1) binding of i-motif DNA to a scaffolding particle, thereby forming a i-motif DNA/scaffolding particle complex; and 2) attachment of CDNs to the complex.

In some aspects, scaffolding particles and/or i-motif DNA that are ready for the binding step are commercially available. Alternatively, as described elsewhere herein, a step of modifying one or both of the i-motif DNA and the scaffolding particle to attach a chemically reactive group may be performed before step 1). Depending on the type of modification, the step of attaching the chemically reactive group is carried out using methodology that is known in the art.

Generally, dry or lyophilized forms of scaffolding material is readily available commercially. The dry material is typically dissolved in an organic solvent that is miscible with water, examples of which include but are not limited to: acetone, acetonitrile, butyric acid, dimethoxyethane, dimethyl sulfoxide, ethylamine, formic acid, various alcohols and diols, propylene glycol, pyridine, etc. Once dissolved, the scaffolding material is added slowly, e.g. dropwise, to an aqueous based medium, which can be water, to form scaffolding nano- or micro-particles. Various steps of stirring, concentrating, washing, etc. may ensue.
Step 1) of the method of making STING-NVs is typically performed by mixing scaffolding particles and i-motif DNA under conditions which permit chemical groups on one or both of the reactants to react and form a bond, usually a covalent bond. For example, for i-motif DNA comprising a free sulfate will react to form a covalent bond with reactive groups of the particles. The reaction may be performed, e.g. such as about 37° C., in a buffer solution (e.g. a physiologically compatible buffer such as PBS at a pH of about 7.0-7.5). The reaction is performed for a period of time sufficient to attach a desired amount of i-motif DNA to the scaffolding particle, as describe elsewhere, to form a i-motif DNA/scaffolding particle complex. Various steps of stirring, concentrating, washing, etc. may ensue prior to step 2).
Step 2) of the method of making STING-NVs is typically performed by mixing a plurality of CDNs with a plurality of scaffolding particle complexes under conditions which permit the attachment of the CDNs to the i-motif DNA that is immobilized at the scaffolding particle complexes (on the surface or inside of the scaffold). The reaction is typically carried out in an aqueous-based buffer at a temperature of e.g. 37° C., for a period of time sufficient to achieve the desired amount of loading. Generally, this step is performed at a pH ranging from about 7 to about 7.4. Various steps of stirring, concentrating, washing, etc. may ensue after step 2) to obtain the product.

Those of skill in the art will recognize that the concentration of CDNs and particulate complexes will also influence the extent of loading, as will the amount of time that the reactants are in contact. These parameters can be readily adjusted by one of skill in the art to achieve a desired level of loading of the STING NVs.

### Exemplary Methods

Additional examples of methods that are encompassed by the present disclosure include but are not limited to:

Methods of treating cancer in a subject (patient) in need thereof. Such methods typically involve administering to the subject a therapeutically effective amount of the STING-NVs disclosed herein.

Also encompassed are methods of increasing the survival time of a subject with cancer. Such methods typically involve administering to the subject a therapeutically effective amount of the STING-NVs disclosed herein.

Also encompassed are methods of decreasing tumor growth in a subject with cancer. Such methods typically involve administering to the subject a therapeutically effective amount of the STING-NVs disclosed herein.

Also encompassed are methods of treating a viral infection in a subject (patient) in need thereof. Such methods typically involve administering to the subject a therapeutically effective amount of the STING-NVs disclosed herein. Therapeutically effective amounts are discussed elsewhere herein.

Also encompassed are methods of stimulating the immune system of in a subject (patient) in need thereof. Such methods typically involve administering to the subject a therapeutically effective amount of the STING-NVs disclosed herein. Therapeutically effective amounts are discussed elsewhere herein.

Also encompassed are methods of delivering CDNs to an immune cell. The methods generally involve contacting the immune cell with at least one STING-NV disclosed herein under conditions and for a period of time sufficient to allow the immune cell to endocytose the STING-NV. Once within the endocytic vacuole, where the pH is low (acidic), the CDNs are released from the STING-NV and then they are released into the microenvironment via endosome escape. The immune cell may be present in a laboratory setting, i.e. the methods are carried out in vitro, e.g. suing cultured immune cells. Alternatively, the immune cell may be present in a host such as a subject or patient in need of immune stimulation. In the latter case, a therapeutically effective amount of the STING-NVs is administered to the subject. Therapeutically effective amounts are discussed elsewhere herein. Immune cells to which the CDNs may be delivered include but are not limited to: macrophages, dendritic cells (DCs), monocytes, natural killer cells, neutrophils, tumor cells, epithelial cells, fibroblasts, etc.

Also encompassed are methods of activating the STING protein. These methods typically comprise a step of contacting the STING protein with at least one CDV that has been released from a STING-NV as disclosed herein under conditions and for a period of time sufficient to allow the CDN to bind to and activate the STING protein. This method may be performed in vitro, e.g. by combining a STING-NV with the STING protein under conditions which permit CDNs to be released from the STING-NV and contact STING (e.g. acidic conditions). Alternatively, the method may be performed in vivo in a subject in need of STING activation. In this case, a therapeutically effective amount of the STING-NVs is administered to the subject, the STING-NVs are endocytosed within the subject, CDNs are released from the STING-NVs and they are then free to bind to STING, thereby activating the protein.

Also encompassed are pH-responsive STING-NVs and methods of their use for CDN delivery to modulate the immune microenvironment, e.g. the tumor immune microenvironment or a viral infection microenvironment, for cancer immunotherapy and/or treatment of viral infections. A described herein, STING-NVs promote CDN delivery into immune cells (such as DCs and macrophages), and mediate pH-responsive CDN release within acidic endosomes upon C-quadruplex formation. C-quadruplex formation leads to CDN dissociation from i-motifs, and the CDNs are released into the surrounding environment by endosome escape, e.g. by pore formation, endosome rupture and/or fusion of the endosome membrane with the membrane of another cell.

### Kits and Packaging

In some aspects, the NVs are incorporated into a sterile concentrated liquid form or a sterile lyophilized form for reconstitution before use. Such forms may be provided to the end user, e.g. in a kit or packaged form. Kits may be supplied to e.g. investigators for research purposes and may include e.g. various buffers for reconstitution, directions for use, labels to attach to the NVs, molecular weight standards, etc. In packaging for use e.g. by a medical professional who will use the formulation to treat a patient, the same or similar components may be included. The NVs may be packaged e.g. in a sterile ampule or bottle if they are in liquid form, or in plastic wrap, a blister pack, etc. if in dry form.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### Example

### Experimental Section

Materials. poly(d,l-Lactide) with poly(ethylene glycol) with maleimide end (MAL-PEG₂₀₀₀-b-PLA₃₀₀₀) was purchased from JenKem Technology. Dithiothreitol (DTT) was purchased from Fisher Scientific. All oligonucleotides (see sequences in Table S1) were purchased from Integrated DNA Technologies. Sodium ascorbate was purchased from Sigma-Aldrich. c-di-GMP (CDG) was purchased from InvivoGen. Fluorescein-labeled CDG was purchased from BioLog Life Science Institute. All other chemicals and solvents were purchased from Sigma-Aldrich (St. Louis, MO) and used without further purification unless otherwise noted.

TABLE S1
The sequence of 3C-i-motif, 5C-i-motif,
7C-i-motif, 9C-i-motif, and scramble
sequence, and the CDG loading efficiency
and loading capacity of these primer
modified NPs.

| | | Loading | Loading |
|---|---|---|---|
| | | effi- | |
| DNA | Sequences | ciency | capacity |
| 3C-i- | TTTTTTCCCTTACCCT | 14% | 0.8% |
| motif | TACCCTTACCC | | |
| | (SEQ ID NO: 1) | | |
| 5C-i- | TTTTTTCCCCCTTACC | 53% | 2.9% |
| motif | CCCTTACCCCCTTACC | | |
| | CCC | | |
| | (SEQ ID NO: 2) | | |
| 7C-i- | TTTTTTCCCCCCCTTA | 24% | 1.3% |
| motif | CCCCCCCTTACCCCCC | | |
| | CTTACCCCCCC | | |
| | (SEQ ID NO: 3) | | |
| 9C-i- | TTTTTTCCCCCCCCCT | 25% | 1.4% |
| motif | TACCCCCCCCCTTACC | | |
| | CCCCCCCTTACCCCCC | | |
| | CCC | | |
| | (SEQ ID NO: 4) | | |
| Scramble | CACCACTCCTCACTCC | 2.3% | 0.1% |
| | TCTCCTCTCTCACACC | | |
| | TTC (SEQ | | |
| | ID NO: 5) | | |

Instrumentation. UV-vis absorption spectra were recorded using the Thermo Scientific GENESYS™ 50 UV-Visible Light Spectrophotometer. Dynamic light scattering (DLS) measurements and the particle zeta potential values were determined using with a Zetasizer Nanoseries (Nano ZS90) instrument. Transmission Electron Microscopy (TEM) was conducted on the JEM-1400 Plus TEM. The samples in aqueous solutions (10 μL) were deposited onto carbon-coated copper grids and were allowed to dry in air overnight. The production of proinflammatory factors IFN-α, IL-6 and IL-12 were determined using with a Synergy H1 Hybrid Multi-Mode Reader (BioTek). Fluorescence images of cancer cells were acquired by the laser scanning microscope Carl Zeiss LSM-710. Binding measurements were conducted using a NanoTemper Monolith NT. Automated Instrument (NanoTemper Technologies GmbH, Germany).
Synthesis of PEG-b-PLA NP. MAL-PEG₂₀₀₀-b-PLA₃₀₀₀ was dissolved in various organic solvents that are miscible with water. NPs were formed by adding the polymer solution dropwise to water. The resulting NP suspension was allowed to stir uncovered for 12 h at room temperature. NPs were purified by ultrafiltration (15 min, 3000 g, Amicon Ultra, Ultracel membrane with 10,000 NMWL, Millipore, Billerica, MA, USA). The PEG-b-PLA NPs were resuspended, washed with water, and collected likewise.
Synthesis of i-motif-NP. Thiol modified i-motif (0.04 mM) was pretreated with DTT (0.1 M) in 1×PBS for 1 h at 37° C. to cleave the dithiol bond, and then NAP5 columns were used to purify the i-motif by removing the DTT and cleaved thiol-appending fragments in 1% sodium ascorbate buffer. The cleaved DNA products were mixed and reacted with MAL-PEG-b-PLA NPs dissolved in 1×PBS for 1 min at room temperature. i-motif-NPs were purified by ultrafiltration (15 min, 3000 g, Amicon Ultra, Ultracel membrane with 10,000 NMWL, Millipore, Billerica, MA, USA). The i-motif-NPs were resuspended, washed with water, and collected likewise.
Preparation of CDG-loaded i-motif-NP. The i-motif-PEG-PLA NPs solution was mixed with the desired amount of CDG to prepare CDG-loaded NPs. The mixture was stirred at 37° C. for 12 h. CDG-loaded i-motif-NPs were purified by ultrafiltration (15 min, 3000 g, Amicon® Ultra, Ultracel membrane with 10,000 NMWL, Millipore, Billerica, MA, USA), and the filtrate was used to calculate drug loading capacity. The resulting products are denoted as STING-NVs. The CDG concentration in the filtrate was determined by a UV-Vis spectrophotometer at 254 nm to calculate the drug loading capacity. The drug loading capacity and loading efficiency were calculated by the following equations:

Loading capacity=(CDG weight in NPs)/(weight of NPs);

Loading efficiency=(CDG weight in NPs)/(total weight of CDG).

CDG release from STING-NVs. First, STING-NVs were dispersed in 1 mL of 1×PBS at different pH and agitated at 200 rpm. Then the mixture was ultra-filtrated at each hour point. The filtrate was taken for CDG detection, and the same volume of fresh buffer was added back to the residual mixture. The amount of released CDG in the filtrate was determined by measuring the absorption at 254 nm using a UV-Vis spectrometer.
Lipo-CDG Synthesis. DOTAP, cholesterol, and DSPE-PEG(2000) were purchased from Avanti Polar Lipids. Lipids were mixed at molar ratios of 1:1:0.2 DOTAP:cholesterol:DSPE-PEG(2000). Lipid mixtures were dried under a nitrogen stream in glass test tubes. The resulting lipid films were placed in a desiccation system overnight. Dry films were hydrated in a solution of 250 μg/mL CDG in DI water with six cycles of vortexing for 30 s every 5 min. Samples were then extruded using 21 passes in a mini-extruder (Avanti) through a 0.2 μm pore size polycarbonate filter (Whatman). Samples were placed in 10K MWCO dialysis cartridges (Thermo Scientific) and dialyzed against PBS for 6 h before use. The CDG content of the liposomes was determined by measuring the absorbance peak of the liposomes at 254 nm, corresponding to the maximum absorbance of CDG, subtracting the lipid contribution to absorbance at this wavelength.
MST and data analysis. MST was carried out in PBST (1×PBS, 0.05% Tween-20) at pH 7.4 or pH 5.0, using NT.Label-Free Standard treated capillaries. 5C-NPs or scramble DNA-NPs (0.5 μM DNA) and increasing CDG concentrations were mixed and incubated for 30 min at room temperature. Samples were then transferred into the NanoTemper Monolith NT.Automated Instrument. MST was carried out at high MST power for the measurements. The MST data of independent measurements were processed using MO. Affinity Analysis v2.3 software to calculate the dissociation constant (K_{d}), for which the fitting (e.g., FIG. 2e) was processed using Sigmoidal (4PL, x is log(concentration)) in Prism GraphPad software.
Cell culture. THP-1 cells were cultured in RPMI 1640 medium (Gibco) supplemented with 10% fetal bovine serum (FBS), 100 U/mL penicillin, 100 μg/ml streptomycin, 2 mM 1-glutamine, 50 μM 2-mercaptoethanol, 1× non-essential amino acids and 10 mM HEPES. DC2.4 cells were cultured in RPMI medium supplemented with 10% FBS, 100 U/mL penicillin, 100 μg/ml streptomycin. B16F10 and RAW264.7 cells were cultured in Dulbecco's Modified Eagle's medium (DMEM) supplemented with 10% FBS, 100 U/mL penicillin, 100 μg/ml streptomycin. All cells were cultured in 37° C. with 5% CO2.
Repolarization of M2 macrophages in vitro. 5×10⁵/mL RAW264.7 cells were cultured in LPS (500 ng/ml) and IFN-γ (25 ng/ml) to induce an M1 phenotype (M1+) or IL-4 (25 ng/ml) to induce an M2 phenotype (M2+). On day 2, M2 cells were treated with indicated formulations for 24 h. On day 3, cells were harvested for gene expression analysis (M1/M2 biomarkers and costimulatory molecules) and flow cytometry; supernatants were collected for cytokine detection (ELISA/Luminex assay).
ELISA. DC2.4 cells were plated at densities of 5×10⁵ cells/well in a 6-well plate. Cells were treated with the indicated formulations for 24 h. Secreted IFN-α was quantified with the LumiKine Xpress mIFN-α enzyme-linked immunosorbent assay kit (InvivoGen). Secreted IL-6 and IL-12 were quantified with the Mouse IL-6 Quantikine™ ELISA Kit (R&D Systems).
Flow Cytometry. In cell uptake experiments, cells were plated at 5×10⁵ cells/mL per well of 6-well plates and treated with various formulations of fluorescein-CDG at 0.5 μg/mL for the indicated time. Cells were analyzed for fluorescein signal (from fluorescein-modified CDG) on the CytoFLEX LX (Beckman Coulter) flow cytometer.
For immune activation studies, cells were stimulated for 24 h with 1.5 μg/mL equivalent CDG, collected and stained for viability and nucleated cells as above, and further stained with Alexa647-conjugated anti-mouse CD80, PerCP-conjugated anti-mouse CD86, FITC-conjugated anti-mouse CD206, and PE-conjugated anti-mouse I-A/I-E before flow cytometry analysis.
Gene Expression Analysis. For in vitro gene expression studies, M1 or M2 Cells were lysed, and RNA was purified using a RNeasy Plus Mini Kit (Qiagen), quantified using a Nanodrop (Thermo Scientific), and transcribed into cDNA using the High-Capacity RNA-to-cDNA™ Kit (Applied Biosystems) according to the manufacturer's instructions. Quantitative real-time PCR (qPCR) for murine costimulatory molecules (Cxcl9 and Cxcl10), M1 markers (Tnf, IL6, IL12, Nos2, and Ifnb1), M2 markers (Ym1, Fizz1, Arg1, and Mrc1) were performed using Power SYBR® Green PCR Master Mix and Power SYBR® Green RT-PCR Reagents Kit (Applied Biosystems). Primer sequences are as follows:

mouse Ifnb1:
Forward Sequence,
(SEQ ID NO: 6)
5′-CGAGCAGAGATCTTCAGGAAC-3′
Reverse Sequence,
(SEQ ID NO: 7)
5′-TCACTACCAGTCCCAGAGTC-3′
mouse Gapdh:
Forward Sequence,
(SEQ ID NO: 8)
5′-CTTTGTCAAGCTCATTTCCTGG-3′
Reverse Sequence,
(SEQ ID NO: 9)
5′-TCTTGCTCAGTGTCCTTGC-3′
mouse Tnf:
Forward Sequence,
(SEQ ID NO: 10)
5′-GGTGCCTATGTCTCAGCCTCTT-3′
Reverse Sequence,
(SEQ ID NO: 11)
5′-GCCATAGAACTGATGAGAGGGAG-3′
mouse Cxc110:
Forward Sequence,
(SEQ ID NO: 12)
5′-ATCATCCCTGCGAGCCTATCCT-3′
Reverse Sequence,
(SEQ ID NO: 13)
5′-GACCTTTTTTGGCTAAACGCTTTC-3′
mouse Cxc19:
Forward Sequence,
(SEQ ID NO: 14)
5′-CCTAGTGATAAGGAATGCACGATG-3′
Reverse Sequence,
(SEQ ID NO: 15)
5′-CTAGGCAGGTTTGATCTCCGTTC-3′
mouse Fizz1:
Forward Sequence,
(SEQ ID NO: 16)
5′-TCCCAGTGAATACTGATGAGA-3′
Reverse Sequence,
(SEQ ID NO: 17)
5′-CCACTCTGGATCTCCCAAGA-3′
mouse Ym1:
Forward Sequence,
(SEQ ID NO: 18)
5′-GGGCATACCTTTATCCTGAG-3′
Reverse Sequence,
(SEQ ID NO: 19)
5′-CCACTGAAGTCATCCATGTC-3′
mouse ARG1:
Forward Sequence,
(SEQ ID NO: 20)
5′-CAGAAGAATGGAAGAGTCAG-3′
Reverse Sequence,
(SEQ ID NO: 21)
5′-CAGATATGCAGGGAGTCACC-3′
mouse NOS2:
Forward Sequence,
(SEQ ID NO: 22)
5′-TGCATGGACCAGTATAAGGCAAGC-3′
Reverse Sequence,
(SEQ ID NO: 23)
5′-GCTTCTGGTCGATGTCATGAGCAA-3′
mouse Mrc1:
Forward Sequence,
(SEQ ID NO: 24)
5′-GTTCACCTGGAGTGATGGTTCTC-3′
Reverse Sequence,
(SEQ ID NO: 25)
5′-AGGACATGCCAGGGTCACCTTT-3′
mouse IL-6:
Forward Sequence,
(SEQ ID NO: 26)
5′-GAGGATACCACTCCCAACAGACC -3′
Reverse Sequence,
(SEQ ID NO: 27)
5′-AAGTGCATCATCGTTGTTCATACA - 3′
mouse IL-12p40:
Forward Sequence,
(SEQ ID NO: 28)
5′-CAGAAGCTAACCATCTCCTGGTTTG-3′
Reverse Sequence,
(SEQ ID NO: 29)
5′-TCCGGAGTAATTTGGTGCTTCACAC-3′

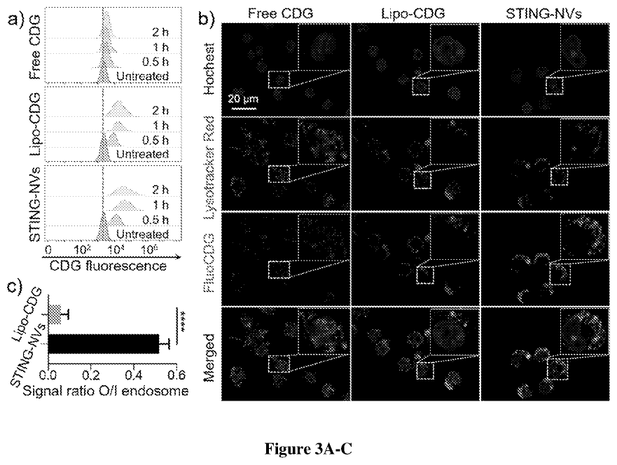

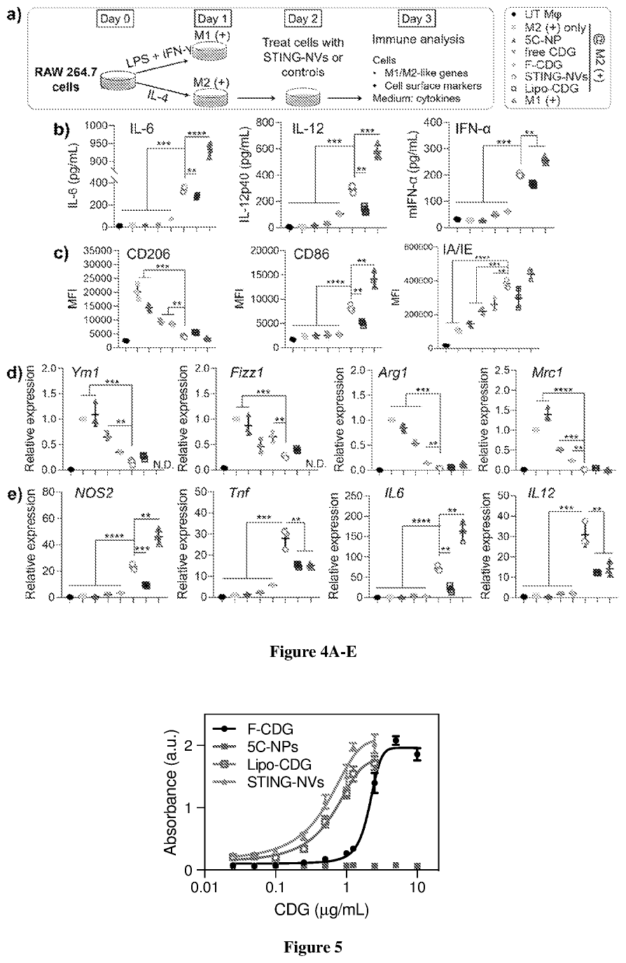

For in vivo gene expression studies, at indicated time points after treatment, tumor tissue samples were collected and placed immediately into RNAlater solution (Thermo Scientific) and stored at 4° C. overnight. Tumor tissues were homogenized using 3.0 mm zirconium. Samples were centrifuged to remove debris and RNA was purified and analyzed as above by qPCR.
In vitro cellular uptake by confocal microscopy. In vitro cell uptake of free CDG, Lipo-CDG, and STING-NVs were studied using confocal fluorescence microscopy. Cells were stained with LysoTracker Red DND-99 (Life Technologies) for 1.5 h and were then stained with L Hoechst33342 (Life Technologies) for 0.5 h and were finally treated with indicated formulation of fluorescein-CDG at 0.5 μg/mL for a specified time length. Cells were then washed with Dulbecco's PBS for three times prior to fluorescence imaging. Samples were imaged using an LSM 710 confocal microscope (Zeiss) at the VCU Microscopy Facility. Endosome escape was studied by quantifying the outside/inside ratio of FluoCDG fluorescence intensities in 10 representative cells using ZEISS Zen software.
Immune activation in THP-1 cells: THP-1 cells were seeded at densities of 1×10⁴ cells/well in a 96-well plate. Cells were treated with CDG formulations (3 μg CDG equivalents) for 18 h. At the same time, HEK-Blue™ IFN-α/p cells (Invivogen) were seeded at densities of 1×10⁴ cells/well in a 96-well plate. Then 100 μL THP-1 cell supernatant was added to HEK-Blue™ IFN-α/D cells for 24 h. 50 μL cell supernatant was collected from each sample and added to 150 μL of QUANTI-Blue™ SEAP detection medium (InvivoGen) and incubated for 2 h at 37° C. SEAP activity was assessed by measuring the absorbance at 630 nm on a plate reader. The results were fit (FIG. 4F) using log(agonist) vs. response (three parameters) in the Prism GraphPad software.
Animal studies. All work conducted on animals were cared for following NIH guidelines and in accordance with an approved protocol by the Virginia Commonwealth University Animal Care and Use Committee (IACUC). Female C57BL/6 mice (6-8 weeks) were purchased from the Charles Rivers.
B16F10 tumor-bearing mice (6-8 weeks old, female) were prepared by subcutaneously injection of 3×10⁵ B16F10 cells in PBS (100 μL). When the tumor was established on day 7 (tumor volume ˜60 mm³) post tumor inoculation, mice started to be treated with specified regimens by intratumoral injection of CDG formulations (3 μg CDG equiv. in 30 μL PBS). Mice were treated for 5 times every 3 days. Tumor tissues were collected to analyze the immune milium in the tumor microenvironment. Tumor volume and mouse weight were monitored every 3 days. Mice were euthanized when any dimension of tumor was close to 2 cm or when mouse body weight was lost by over 20%. Tumor volume was calculated using the following formula:

Volume=(length*width²)/2

Statistical Analyses. Data were analyzed by nonparametric unpaired t tests (Mann-Whitney) or one-way ANOVA followed by Dunnett's multiple comparison tests using GraphPad Prism. p<0.05 was considered statistical significance.

### Synthesis and Characterization of STING-NVs

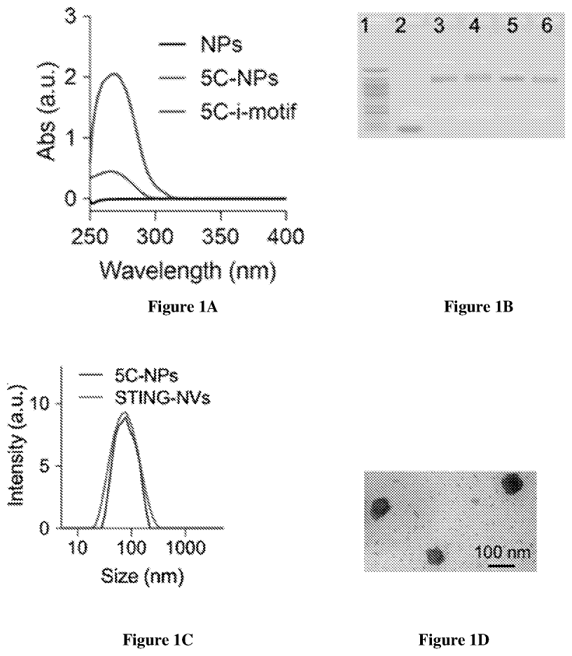

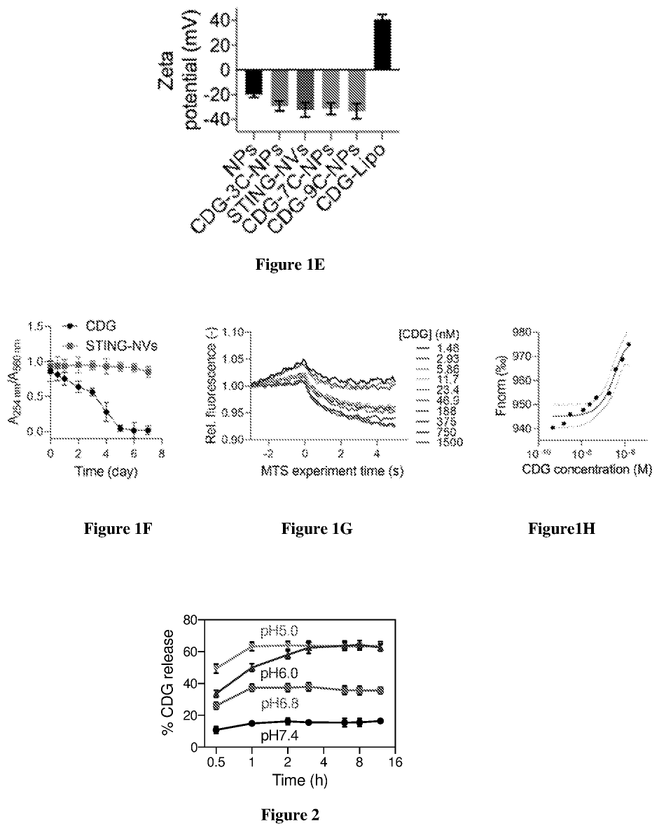

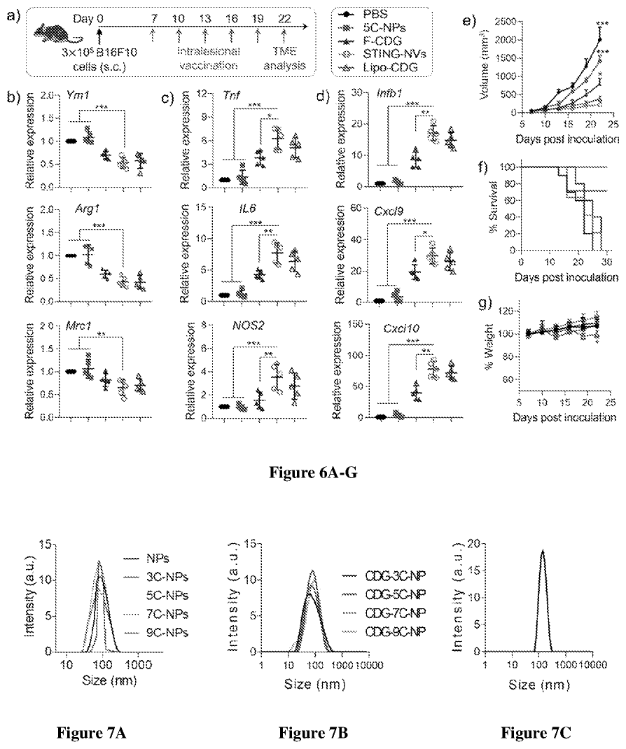

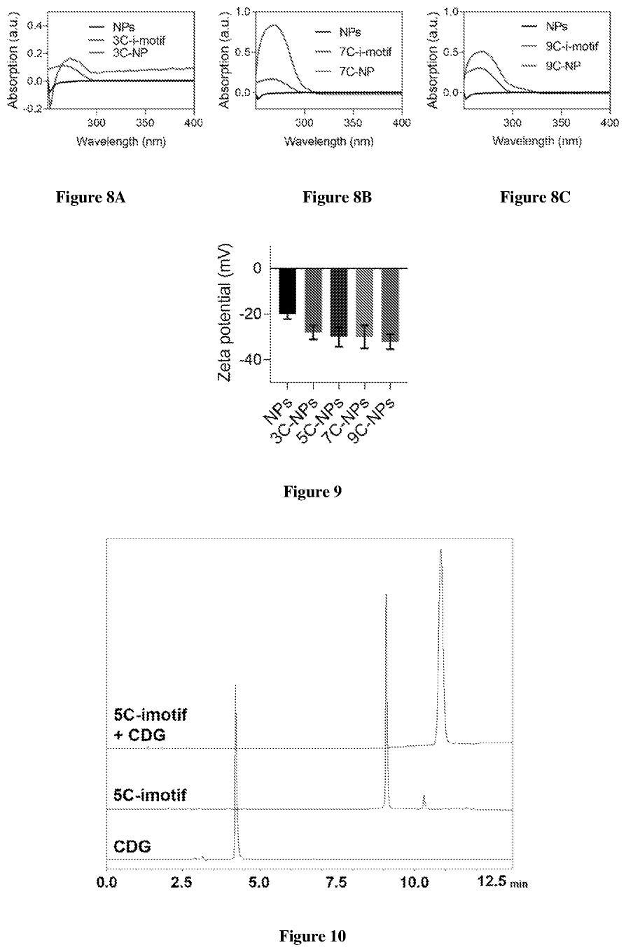

To synthesize the core of STING-NVs, we used biocompatible amphiphilic diblock copolymer PEG-b-PLA to self-assemble micellular NPs with hydrodynamic diameters of 78±35 nm in water solution as measured by dynamic light scattering (DLS) (FIG. 7A). Next, thiol-modified i-motif DNA, which consisted of 4 segregated domains of consecutive Cs (sequences in Table S1), were conjugated with the terminal maleimide of PEG-b-PLA on the surfaces of the above NPs. To optimize the lengths of i-motifs for CDG loading, we synthesized NPs with four different i-motifs that respectively have 3, 5, 7, and 9 consecutive Cs in each C domain, as well as a scramble DNA as a control (Table S1). UV-vis absorption spectra and gel electrophoresis verified the conjugation of DNA with PEG-b-PLA NPs (FIG. 1A, 1B and FIG. 8A-C). The resulting DNA-modified NPs have hydrodynamic sizes of 80-90 nm (FIGS. 7B and B). The zeta potential of i-motif-NPs (ca. −30 mV) decreased relative to PEG-b-PLA NPs (ca. −20 mV) (FIG. 9), which further verified the conjugation of negatively charged DNA on NPs. To optimize i-motif lengths for CDG loading, CDG was incubated with the above five DNA-modified NPs, respectively, followed by purification via centrifuge filtration and quantification of loaded CDG in the NPs by UV-vis spectrometry. As a result, 5C-i-motif-modified NPs (5C-NPs) exhibited the highest loading efficiency (53%±1%) and loading capacity (2.93%±0.17% w/w) (Table S1). Therefore, 5C-NPs and the corresponding CDG-loaded NPs (thereafter termed as STING-NVs) were used for the following studies. The hydrodynamic diameters of STING-NVs were 81±36 nm (FIG. 1C), as verified by transmission electron microscope (TEM) (FIG. 1D). Due to the negative charges of i-motifs and CDG, the zeta potential of all CDG-loaded i-motif-NPs showed strong negative charge, whereas a control of CDG-loaded cationic liposomes (Lipo-CDG) was still positive charged (FIG. 1E). Note that a 20:1 of PEG-b-PLA to 5C-i-motif molar ratio in the initial reaction yielded the highest loading efficiency (53%±1%), and this ratio was used for further studies (Table S2).

TABLE S2
The i-motif conjugation efficiency, CDG loading efficiency
and loading content of the different molar ratio of 5C-
i-motif to PEG-b-PLA in the initial reaction mixture.

| | Conjugation | Loading | | |
|---|---|---|---|---|
| n(PEG-b-PLA):n(5C- | effi- | effi- | Loading | n(CDG):n(5C- |
| i-motif) | ciency | ciency | content | i-motif) |
| 2.5:1 | 81% | 8% | 3.2% | 0.63:1 |
| 5:1 | 92% | 21% | 4.2% | 1.3:1 |
| 10:1 | 85% | 25% | 2.6% | 1.7:1 |
| 20:1 | 93% | 53% | 2.9% | 4.3:1 |
| 50:1 | 87% | 47% | 0.9% | 3.4:1 |

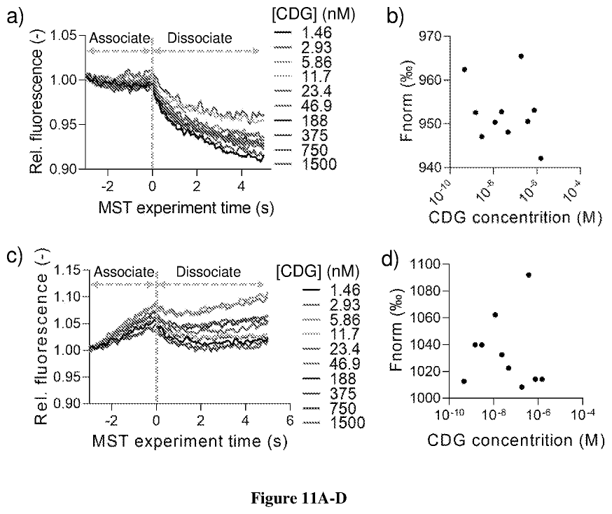

Interestingly, STING-NVs dramatically improved the biostability of CDG. After incubation in cell culture medium supplemented with 10% serum for 7 days, STING-NVs showed 91.4% intact CDG as determined by the characteristic absorption of CDG at 254 nm, in contrast to only 2.3% for free CDG (FIG. 1F). The ability of STING-NVs to promote the biostability of CDG is presumably due to the steric hindrance of i-motif-NPs that prevent the access of nucleases. Using HPLC and microscale thermophoresis (MST), we further studied the binding of CDG with 5C-i-motifs, which is the basis for CDG loading into i-motif-modified NPs. In HPLC, relative to free CDG and 5C-i-motif, the delayed elution time of physically mixed CDG and 5C-i-motif indicated the complexation of CDG and 5C-i-motif (FIG. 10). By MST, the binding affinity (K_{d}) of CDG with 5C-NPs was determined to be 47 nM (FIG. 1G, 1H), in contrast to negligible binding of CDG with scramble-NPs (FIGS. 11A and 11B). The strong and specific binding between CDG and 5C-NPs provided the foundation for efficient CDG loading in 5C-NPs.
pH-Responsive Cumulative CDG Release from STING-NVs

Next, we evaluated the pH-responsive CDG release from STING-NVs. We hypothesized that under acidic condition, the formation of i-motif C-quadruplexes via protonated C:C+ base pairing competes with CDG binding with the Cs in i-motifs, leading to CDG release from the i-motifs in STING-NVs. By MST, we verified negligible binding of CDG with 5C-NPs at pH 5.0 (FIGS. 11C and 11D). A release study in different pH at 37° C. showed that slightly acidic condition (pH 6.8) already significantly promoted CDG burst release from STING-NVs, and CDG release was further facilitated by lowering the pH down to pH 5.0 (FIG. 2). Specifically, at pH 5.0, the CDG release half-lives from 3C-NPs, i0 STING-NVs, 7C-NPs, and 9C-NPs is 0.10 h, 0.13 h, 0.14 h, and 0.16 h, respectively. CDG release from STING-NVs was increased by over 5 times within 1 h at pH 5.0 (˜65%) relative to pH 7.4 (˜12%). CDG release plateaued at 55-65%, presumably due to the equilibrium of CDG: i-motif binding and C-quadruplex formation. These results provide the basis for conditional CDG release from STING-NVs in the acidic endosome of immune cells.

### STING-NVs Promoted the Intracellular Delivery of CDG and Facilitated Endosome Escape of CDG in Macrophages.

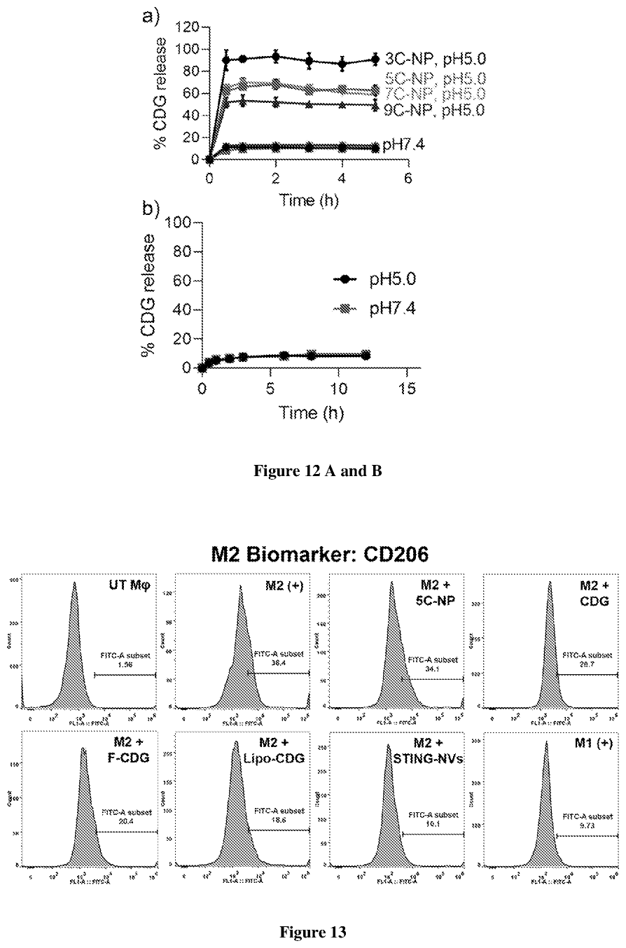

We then studied the intracellular delivery of STING-NVs in macrophages, which are commonly involved in STING activation, with free CDG and Lipo-CDGU^{[13]} as controls. Fluorescein-conjugated CDG (FluoCDG) was used to monitor CDG uptake by flow cytometry and fluorescence confocal laser scanning microscopy (CLSM). RAW264.7 murine macrophages treated with free CDG showed only slight FluoCDG signal enhancement over 2 h; in contrast, cells treated with Lipo-CDG and STING-NVs showed significant FluoCDG fluorescence signal enhancement 0.5 h post incubation, which reached an order of magnitude higher FluoCDG fluorescence signal intensities than untreated cells within 1 h (FIG. 3A). Interestingly, the intracellular FluoCDG signal of cells treated with STING-NVs outperformed cells treated with cationic Lipo-CDG that is typically efficient at intracellular delivery. This is presumably due to the less efficient release [only 10% CDG release from Lipo-CDG in 12 h (FIGS. 12A and B)] and less efficient endosome escape of CDG in Lipo-CDG than CDG in STING-NVs (FIG. 3B, 3C). Indeed, CLSM revealed massive colocalization of endolysosome with STING-NVs as well as Lipo-CDG, respectively (FIG. 3B). Remarkably, the ratio of intracellular FluoCDG fluorescence signal intensities outside over inside endolysosome is 0.52 for STING-NVs, which is nearly nine times of the ratio for Lipo-CDG (0.06) (FIG. 3C). These results indicated that STING-NVs mediated efficient endosome escape of CDG, which is essential for CDG to activate STING that is located in the endothelium reticulum.

### Potent Immunostimulation by STING-NVs

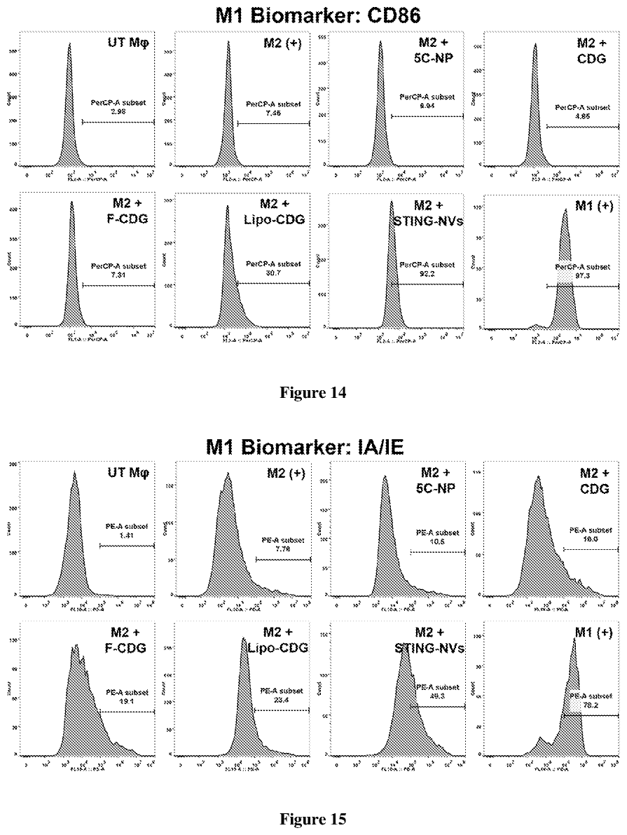

Given efficient intracellular delivery and endosome escape of STING-NVs, we then evaluated STING-NVs for STING activation in macrophages and DCs, both of which are critical for tumor immunotherapy. Given the ability of CDNs to repolarize protumorigenic M2-like phenotype to antitumor M1-like phenotype in mouse and human macrophages,^{[8c]} we studied STING-NVs for macrophage repolarization using a model of murine macrophage. Specifically, M2-like macrophages were generated by treating RAW264.7 murine macrophages with interleukin-4 (IL-4) (FIG. 4A). As a control, M1-like macrophages were generated by treating RAW264.7 cells with lipopolysaccharide (LPS) and IFN-γ. In M2 cells, STING-NVs treatment for 24 h significantly enhanced the secretion of proinflammatory factors IL-6, IL-12p40, and IFN-α, compared to free CDG and F-CDG (FIG. 4B). Moreover, immunostaining and flow cytometry revealed that, STING-NVs significantly upregulated the expression of M1 markers MHC class II (I-A/I-E) and costimulatory factor CD86 and downregulated the expression of M2 marker CD206 (FIG. 4C, FIG. 13, FIG. 14 and FIG. 15). Consistently, as shown by quantitative reverse transcription PCR (RT-qPCR), compared to free CDG, F-CDG, and 5C-NPs, STING-NV treatment in M2 cells significantly decreased the mRNA levels of M2 gene markers Ym1, Fizz1, Arg1, and Mrc1 (FIG. 4D, 4E), and significantly increased the mRNA levels of M1 gene markers Nos2, Tnf, IL6, and IL12b (FIG. 4E). These results indicated that STING-NVs promoted the ability of CDG to repolarize M2-like macrophage to M1-like cells, which is expected to elicit innate and adaptive antitumor immune responses. Worth noting, STING-NVs outperformed Lipo-CDG, which is perhaps one of the current gold standards of CDN carriers, for immunostimulation (FIG. 4).

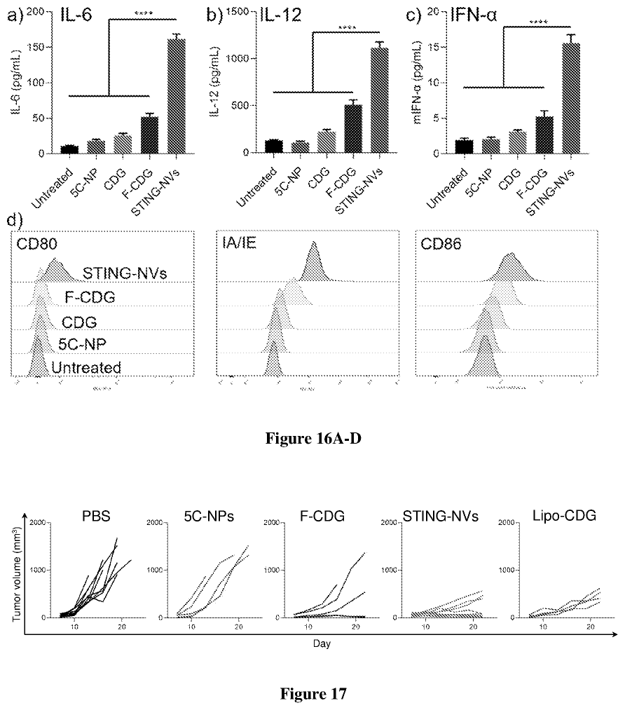

We further studied STING-NVs for immunostimulation in DCs using murine DC2.4 cells. Cells were again treated with STING-NVs for 24 h, followed by flow cytometric analysis of cell surface expression levels of MHC class II (IA/IE) and costimulatory factor CD80 and CD86, as well as ELISA analysis of IL-6, IL-12p40, and IFN-α in cell culture medium. Consistent with the studies in macrophages, compared with free CDG and F-CDG, STING-NV treatment in DC2.4 cells significantly promoted the secretion of IL-6, IL-12p40, and IFN-α (FIG. 16A-C). Further, STING-NVs elevated the expression levels of MHC-II, CD86, and CD80 on DC2.4 cells, relative to soluble CDG or CDG-F (FIG. 16D). Given the discrepancy of the responsiveness of human and murine STINGs to agonists, we also evaluated STING-NVs to activate type I IFN responses in human monocytes (FIG. 5). Specifically, in human THP-1 cells, STING-NVs dramatically promoted IFN-α/β production in THP-1 cells, especially compared to F-CDG. Overall, STING-NVs showed potent immunostimulatory efficacy in mouse and human immune cells.

### STING-NVs Modulated the Immune Milieu in TME and Mediated Potent Immunotherapy

Encouraged by the above in vitro results, we then studied STING-NVs to modulate the tumor immune microenvironment in poorly immunogenic B16F10 melanoma tumors in syngeneic C57BL/6 mice. Subcutaneous tumors (˜60 mm³) were intratumorally administered with STING-NVs, with controls of PBS, CDG-F, 5C-NPs, and Lipo-CDG at a low dose of 3 μg every three days for 5 times (FIG. 6A). 3 days after the last treatment, tumor tissues were harvested, homogenized, and lysed, followed by using RT-qPCR to analyze the tumor immune milieu related mRNA transcript levels in the TME. As a result, relative to all control regimens, STING-NVs significantly decreased M2-associated gene transcripts Ym1, Arg1, and Mrc1 (FIG. 6B), and increased M1-associated gene transcripts Tnf, IL6, and Nos2 (FIG. 6C). Moreover, STING-NVs also increased the expression of Ifnb1, Cxcl9, and Cxcl10 (FIG. 6D), which are critical mediators of antitumor T cell activation and recruitment.^{[8a, 9a, 14]} These results demonstrate the ability of STING-NVs to repolarize macrophages from an M2-like phenotype toward an M1-like phenotype and promote the activation of antitumor T cell responses in vivo. Overall, STING-NVs showed the ability to reprogram tumor immune milieu to reduce immunosuppression in vivo, which is critical for tumor immunotherapy.

Given the ability of STING-NVs to remodel the TME, we studied STING-NVs for tumor immunotherapy in the above B16F10 melanoma mouse model in C57BL/6 mice. STING-NVs were administered intralesionally, as typically performed in current clinical studies of CDN-based immunotherapy. With the same treatment scheme as above (FIG. 6A), STING-NVs significantly reduced tumor growth (FIG. 6E) and prolonged mouse survival (FIG. 6F), compared with blank 5C-NPs and F-CDG. Worth noting, STING-NVs showed the trend of superior therapeutic efficacy and survival benefit than Lipo-CDG, presumably due to the pH-responsive CDG release and efficient endosome escape. Remarkably, after STING-NV treatment, 5/10 melanoma tumors showed no significant tumor growth within the observation period (FIG. 17), whereas F-CDG only slowed tumor growth. Further, no significant body weight changes in STING-NVs treatment were observed over the study period of 22 days, indicating good biocompatibility of these STING-NVs (FIG. 6G). By contrast, treatment with cationic Lipo-CDG led to significant weight lose despite its ability to slow down tumor growth. The ability of STING-NVs to significantly reduce tumor growth rate and prolong mouse survival without significant toxicity in a poorly immunogenic melanoma mouse model demonstrated the potential of intralesional STING-NVs for potent tumor immunotherapy.

### CONCLUSION

In summary, by leveraging the pH-conditional structural reconfiguration of C-rich DNA i-motif and the resulting pH-responsive interaction between CDG and i-motifs, we developed pH-responsive STING-NVs for CDG delivery to modulate tumor immune microenvironment for cancer immunotherapy. STING-NVs efficiently loaded CDG at physiological pH, and dramatically protected CDG from enzymatic degradation. STING-NVs promoted CDG delivery into immune cells such as DCs and macrophages, and mediated pH-responsive CDG release in the acidic endosome upon C-quadruplex formation that led to CDG dissociation from i-motifs. Interestingly, STING-NVs facilitated the endosome escape of CDG into cytosol, which allow CDG to access and activate STING in the endothelium reticulum. In both mouse and human immune cells (macrophages, DCs, or monocytes), STING-NVs elicited potent immune responses, including type I IFN responses which are characteristic of STING activation. STING-NVs repolarized immunosuppressive M2-like macrophages into antitumor M1-like macrophages in a macrophage model in vitro. This was recapitulated in vivo, where STING-NVs repolarized M2-like macrophages toward M1-like macrophages in the TME of a poorly immunogenic melanoma model. Specifically, STING-NVs down-regulated M2 gene markers and up-regulated M1 gene markers in vitro and in vivo, and also promoted antitumor T cell activation and recruitment in vivo. In a poorly immunogenic melanoma model in syngeneic mice, STING-NVs showed superior immunotherapeutic efficacy relative to F-CDG and Lipo-CDG, which are perhaps the current gold standards of CDNs. Taken together, STING-NVs represent a novel CDG delivery system featuring efficient CDG loading presumably via G:C base pairing and conditional CDG release in acidic endosomes of immune cells; STING-NVs exhibited potent immunostimulation in vitro and in vivo, resulting in potent immunotherapy with undetectable side effects in an aggressive melanoma mouse model. These results suggest that STING-NVs expand the armamentarium to promote the efficacy of cancer immunotherapy.

### REFERENCES

[1] a) J. Couzin-Frankel, Science 2013, 342, 1432-1433; b) I. Mellman, G. Coukos, G. Dranoff, Nature 2011, 480, 480-489; c) A. Ribas, J. D. Wolchok, Science 2018, 359, 1350-1355.
[2] P. Darvin, S. M. Toor, V. Sasidharan Nair, E. Elkord, Exp. Mol. Med. 2018, 50, 165.
[3] a) G. N. Barber, Nat. Rev. Immunol. 2015, 15, 760-770; b) L. Zitvogel, L. Galluzzi, O. Kepp, M. J. Smyth, G. Kroemer, Nat. Rev. Immunol. 2015, 15, 405-414; c) Y. Zhang, S. Lin, X. Y. Wang, G. Zhu, Wiley Interdiscip. Rev. Nanomed. Nanobiotechnol. 2019, 11, e1559; d) X. Cen, G. Zhu, J. Yang, J. Yang, J. Guo, J. Jin, K. S. Nandakumar, W. Yang, H. Yin, S. Liu, K. Cheng, Adv. Sci. 2019, 6, 1802042; e) H.-C. Chang, Z. Z. Zou, Q. H. Wang, J. Li, H. Jin, Q. X. Yin, D. Xing, Adv. Sci. 2020, 7, 1900069; f) A. F. U. H. Saeed, X. Ruan, H. Guan, Ji. Su, S. Ouyang, Adv. Sci. 2020, 7, 1902599; g) R. J. Mancini, J. K. Tom, A. P. Esser-Kahn, Angew. Chem. Int. Ed. 2014, 53, 189-192; Angew. Chem. 2014, 126, 193-196.
[4] a) H. Ishikawa, G. N. Barber, Nature 2008, 455, 674-678; b) H. Ishikawa, Z. Ma, G. N. Barber, Nature 2009, 461, 788-792; c) T. Su, Y. Zhang, K. Valerie, X.-Y. Wang, S. Lin, G. Zhu, Theranostics 2019, 9, 7759-7771. [5] a) B. C. Baguley, L.-M. Ching, BioDrugs 1997, 8, 119-127; b) J. M. Ramanjulu, G. S. Pesiridis, J. Yang, N. Concha, R. Singhaus, S. Y. Zhang, J. L. Tran, P. Moore, S. Lehmann, H. C. Eberl, M. Muelbaier, J. L. Schneck, J. Clemens, M. Adam, J. Mehlmann, J. Romano, A. Morales, J. Kang, L. Leister, T. L. Graybill, A. K. Charnley, G. Ye, N. Nevins, K. Behnia, A. I. Wolf, V. Kasparcova, K. Nurse, L. Wang, Y. Li, M. Klein, C. B. Hopson, J. Guss, M. Bantscheff, G. Bergamini, M. A. Reilly, Y. Lian, K. J. Duffy, J. Adams, K. P. Foley, P. J. Gough, R. W. Marquis, J. Smothers, A. Hoos, J. Bertin, Nature 2018, 564, 439-443; c) O. Danilchanka, J. J. Mekalanos, Cell 2013, 154, 962-970; d) S. Chattopadhyay, Y.-H. Liu, Z.-S. Fang, C.-L. Lin, J.-C. Lin, B.-Y. Yao, C.-M. J. Hu, Nano Lett. 2020, 20, 2246-2256; e) L. C.-W. Lin, C.-Y. Huang, B.-Y. Yao, J.-C. Lin, A. Agrawal, A. Algaissi, B.-H. Peng, Y.-H. Liu, P.-H. Huang, R.-H. Juang, Y.-C. Chang, C.-T. Tseng, H.-W. Chen, C.-M. J. Hu, Adv. Funct. Mater. 2019, 29, 1807616; f) J. Wang, P. Li, Y. Yu, Y. Fu, H. Jiang, M. Lu, Z. Sun, S. Jiang, L. Lu, M. X. Wu, Science 2020, 367, eaau0810.
[6] a) D. L. Burdette, K. M. Monroe, K. Sotelo-Troha, J. S. Iwig, B. Eckert, M. Hyodo, Y. Hayakawa, R. E. Vance, Nature 2011, 478, 515-518; b) J. J. Woodward, A. T. Iavarone, D. A. Portnoy, Science 2010, 328, 1703-1705; c) A. Ablasser, M. Goldeck, T. Cavlar, T. Deimling, G. Witte, I. Rohl, K. P. Hopfner, J. Ludwig, V. Hornung, Nature 2013, 498, 380-384.
[7] L. Corrales, L. H. Glickman, S. M. McWhirter, D. B. Kanne, K. E. Sivick, G. E. Katibah, S. R. Woo, E. Lemmens, T. Banda, J. J. Leong, K. Metchette, T. W. Dubensky, Jr., T. F. Gajewski, Cell Rep. 2015, 11, 1018-1030.
[8] a) S. T. Koshy, A. S. Cheung, L. Gu, A. R. Graveline, D. J. Mooney, Adv. Biosyst. 2017, 1, 1600013; b) M. C. Hanson, M. P. Crespo, W. Abraham, K. D. Moynihan, G. L. Szeto, S. H. Chen, M. B. Melo, S. Mueller, D. J. Irvine, J. Clin. Invest. 2015, 125, 2532-2546; c) N. Cheng, R. Watkins-Schulz, R. D. Junkins, C. N. David, B. M. Johnson, S. A. Montgomery, K. J. Peine, D. B. Darr, H. Yuan, K. P. McKinnon, Q. Liu, L. Miao, L. Huang, E. M. Bachelder, K. M. Ainslie, J. P. Ting, JCI Insight 2018, 3, e120638.
[9] a) D. Shae, K. W. Becker, P. Christov, D. S. Yun, A. K. R. Lytton-Jean, S. Sevimli, M. Ascano, M. Kelley, D. B. Johnson, J. M. Balko, J. T. Wilson, Nat. Nanotechnol. 2019, 14, 269-278; b) L. C. Lin, S. Chattopadhyay, J. C. Lin, C. J. Hu, Adv. Healthc. Mater. 2018, 7, e1701395.
[10] M. An, C. Yu, J. Xi, J. Reyes, G. Mao, W. Z. Wei, H. Liu, Nanoscale 2018, 10, 9311-9319.
[11] E. Lee, H. E. Jang, Y. Y. Kang, J. Kim, J. H. Ahn, H. Mok, Acta. Biomater. 2016, 29, 271-281.
[12] a) S. Modi, G. S. M, D. Goswami, G. D. Gupta, S. Mayor, Y. Krishnan, Nat. Nanotechnol. 2009, 4, 325-330; b) H. Liu, Y. Xu, F. Li, Y. Yang, W. Wang, Y. Song, D. Liu, Angew. Chem. Int. Ed. 2007, 46, 2515-2517; Angew. Chem. 2007, 119, 2567-2569; c) J. Choi, S. Kim, T. Tachikawa, M. Fujitsuka, T. Majima, J. Am. Chem. Soc. 2011, 133, 16146-16153; d) D. He, X. He, K. Wang, J. Cao, Y. Zhao, Adv. Funct. Mater. 2012, 22, 4704-4710; e) Y. Dong, Z. Yang, D. Liu, Acc. Chem. Res. 2014, 47, 1853-1860.
[13] a) P. Midoux, C. Pichon, Expert. Rev. Vaccines 2015, 14, 221-234; b) A. J. Mukalel, R. S. Riley, R. Zhang, M. J. Mitchell, Cancer Lett. 2019, 458, 102-112; c) V. Colapicchioni, S. Palchetti, D. Pozzi, E. S. Marini, A. Riccioli, E. Ziparo, M. Papi, H. Amenitsch, G. Caracciolo, J. Mater. Chem. B 2015, 3, 7408-7416; d) L. Wasungu, D. Hoekstra, J. Control Release 2006, 116, 255-264; e) L. M. Kranz, M. Diken, H. Haas, S. Kreiter, C. Loquai, K. C. Reuter, M. Meng, D. Fritz, F. Vascotto, H. Hefesha, C. Grunwitz, M. Vormehr, Y. Husemann, A. Selmi, A. N. Kuhn, J. Buck, E. Derhovanessian, R. Rae, S. Attig, J. Diekmann, R. A. Jabulowsky, S. Heesch, J. Hassel, P. Langguth, S. Grabbe, C. Huber, O. Tureci, U. Sahin, Nature 2016, 534, 396-401.
[14] L. Corrales, S. M. McWhirter, T. W. Dubensky, Jr., T. F. Gajewski, J. Clin. Invest. 2016, 126, 2404-2411.

While the invention has been described in terms of its several exemplary embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A drug delivery system for delivering stimulator of interferon genes (STING) agonists, comprising
nano- or microparticulate scaffolds;
synthesized cytosine-rich i-motif DNA on the nano- or microparticulate scaffolds; and
cyclic dinucleotides (CDNs) bound to the cytosine-rich i-motif DNA.

2. The drug delivery system of claim 1, wherein the nano- or microparticulate scaffolds comprise poly(D,L-lactide)-block-poly(ethylene glycol).

3. The drug delivery system of claim 1, wherein the cytosine-rich i-motif DNA comprises four cytosine-rich domains.

4. The drug delivery system of claim 1, wherein the cytosine-rich i-motif DNA comprises 3-9 consecutive cytosines in each cytosine-rich domain.

5. The drug delivery system of claim 1, wherein the CDNs comprise one or more of cyclic dimeric guanosine monophosphate (CDG), cyclic dimeric adenosine monophosphate (CDA), and cyclic GMP-AMP (cGAMP).

6. The drug delivery system of claim 1, wherein a molar ratio of polymer in the nano- or microparticulate scaffolds to the cytosine-rich i-motif DNA is from 18:1 to 22:1.

7. The drug delivery system of claim 1, wherein the nano- or microparticulate scaffolds have an average diameter of 70-100 nm.

8. The drug delivery system of claim 1, wherein the cytosine-rich i-motif DNA is configured to release the CDNs at a pH of 6.8 or lower.

9. A method for delivering STING agonists to a subject in need thereof, comprising administering to the subject a therapeutically effective amount of the drug delivery system of claim 1.

10. The method of claim 9, wherein the subject has cancer.

11. The method of claim 10, wherein the cancer is melanoma.

12. The method of claim 9, wherein the subject has a viral infection.
